(19) United States
(12) Patent Application Publication
(10) Pub. No.: US 2025/0061966 A1
(43) Pub. Date: Feb. 20, 2025

(54) METHODS AND SYSTEMS FOR ASSESSING IMMUNE CELL RECEPTORS AND ANTIGENS

(71) Applicant: 3T BIOSCIENCES, INC., South San Francisco, CA (US)

(72) Inventors: Marvin Gee, San Francisco, CA (US); Norman Goodacre, South San Francisco, CA (US); Eric Luxenberg, South San Francisco, CA (US); Leah Sibener, San Francisco, CA (US)

(21) Appl. No.: 18/720,224

(22) PCT Filed: Dec. 15, 2022
(86) PCT No.: PCT/US2022/053076

§ 371 Date: Jun. 14, 2024

Related U.S. Application Data
(60) Provisional application No. 63/289,729, filed on Dec. 15, 2021.

(51) Int. Cl. G16B 20/30; G01N 33/569; G01N 33/68; G16B 15/30

(52) U.S. Cl. CPC G16B 20/30; G01N 33/56972; G01N 33/6878; G16B 15/30; G01N 2333/70539

(57) ABSTRACT

Described herein are methods and systems used to provide an exhaustive cross-reactivity profile for a TCR.

### FIELD OF DISCLOSURE

The disclosure relates to methods and systems for assessing immune cell receptors.

### BACKGROUND

T cells are the central mediators of adaptive immunity, through both direct effector functions and coordination and activation of other immune cells. Each T cell expresses a unique T cell receptor (TCR), selected for the ability to bind to major histocompatibility complex (MHC) molecules presenting peptides. TCR recognition of peptide-MHC (pMHC) drives T cell development, survival, and effector functions. The peptide-Major Histocompatibility Complex (pMHC) is a non-covalent complex of 3 proteins. In order to improve stability, the pMHC can be constructed as a single chain trimer (SCT), a single fusion protein with the general structure of P-L1-B-L2-A, where L1 and L2 are flexible linkers, P is a target peptide (i.e., peptide ligand), and in the case of MHC Class I, A is a soluble form of the alpha chain of MHC I, and B is beta-2-microglobulin (Yu Y et al. 2002 J Immunol. 168: 3145-9). In SCTs derived from MHC Class I, the Y84A mutation can be introduced into the MHC-alpha domain to better accommodate Linker 1 at the C terminus of the target peptide (i.e., peptide ligand) (Lybarger L et al. 2003 J. Biol. Chem. 278: 27104-11).

The SCT has been adapted for display on the surface of yeast for both MHC Class I and MHC Class II through the fusion to a yeast cell wall protein (e.g., Aga2) (Adams J J et al. 2011 Immunity 35: 681-93; Birnbaum M E et al. 2014 Cell 157: 1073-87; Gee M et al. 2018 Cell 172: 549-63). For MHC Class I, the yeast-displayed SCT has the general structure of P-L1-B-L2-A-L3-T, where T is a yeast cell wall protein (e.g., Aga2), L3 is a flexible linker, and P, B, A, L1 and L2 are as described previously.

Peptide libraries in yeast-displayed SCT of MHC Class I and of Class II have enabled the de-orphanizing of a T cell receptor (TCR) through the identification of the cognate pMHC towards which the TCR is reactive, and identification of off-target cross reactivities to other pMHC (Birnbaum, 2014; Gee, 2018). In many cases, the off-target cross-reactive pMHCs are non-homologous to the intended pMHC target, suggesting that these libraries can more comprehensively identify reactive peptides than other methods that rely on sequence similarity.

This high-throughput data from these aforementioned yeast display libraries provides a reservoir of information amenable to state-of-the-art computational and laboratory techniques to elucidate TCR antigens, activators, cross-reactivities, and general safety profiles.

### SUMMARY

The present invention provides methods and systems that may identify potential antigens that bind to a particular T cell receptor (TCR), determine the immunogenicity of the potential antigens to activate the TCR, and exhaustively profile the receptor in terms of on-target and off-target reactivity to the potential antigens. As a result, the methods and systems of the disclosure are able to identify the specificity of a TCR, including “orphan” TCRs (TCRs of unknown antigen specificity), for particular antigens. The systems and methods are also able to predict efficacy and safety profile of immune cells activated by particular antigens.

TCRs and their cognate peptide-HLA targets, e.g., peptide-Major Histocompatibility Complexes (pMHC), possess inherent variability across receptors and antigens. The variability among the components of various TCR-pMHC systems means that determining the antigen specificity for a TCR has poses a complex problem. Although previous efforts have improved the ability to characterize TCR sequences, the ability to profile the antigen specifies of TCRs and T cells has been a bottleneck in developing TCR-based immunotherapies.

The presently disclosed systems and methods provide a high-throughput path through this bottleneck. By combining computational and wet lab techniques, the systems and methods of the invention provide an unprecedentedly accurate and exhaustive profile of TCR activity and specificity. This includes predicting binding and activity of particular T cell receptors to determine their specificities and/or cross-reactivities to antigens and may help identify novel target antigens and their cross-reactivities to other compounds or receptors. As explained in greater detail herein, the systems and methods of the invention may provide direct identification of antigens that bind to a TCR, including an “orphan” TCR, without any apriori knowledge of the antigens.

The presently disclosed systems and methods have three main components or modules. Each of these components may include one or more wet lab steps and/or one or more computational steps, which preferably include a machine learning system. The first component or module predicts target antigens, e.g., cognate peptide-HLAs, for a particular TCR. This module can similarly predict target antigens of, for example, a TCR mimetic (TCRm). The identified peptide sequences may be used to generate a machine learning model that compares the peptide sequences with, for example, one or more proteome databases to predict potential endogenous ligands of the TCR. The first module may also use the peptide sequence to identify sequence or binding motifs in the peptide sequences, which may identify superbinders—peptide sequences with a higher affinity for a TCR than endogenous antigen.

The second module determines the immunogenicity of peptides to activate a particular TCR of interest, e.g., an orphan TCR. The second module uses T-cell/TCR activation data to train a machine learning model. Once trained, the model may be used to predict the activation potential for peptides or antigens identified by module 1.

The third module is used to exhaustively profile a TCR of interest in terms of on-target and off-target cross reactivity. This module may include constructing an activation neighborhood in a latent space, which may be continually updated to reveal new activators until the cross-reactivity profile of the TCR is determined based on an absence of any new activators.

In certain aspects, the present invention provides methods for profiling a TCR. An exemplary method includes conducting one or more analyses to obtain a plurality of peptide binding predictions that are representative of peptide-HLA targets that a TCR binds; conducting one or more analyses using the peptide binding predictions to obtain a plurality of peptide activation predictions that are representative of the identified target peptides that are predicted to activate the TCR; and conducting one or more analyses using the peptide binding predictions and the peptide activation predictions to obtain a cross-reactivity profile of the TCR with respect to the target peptides identified from the activation predictions to thereby obtain a profile of the TCR.

The step of conducting one or more analyses to obtain a plurality of peptide binding predictions may include: contacting a screening library of cells with a TCR or other macromolecule having one or more antigen binding domains, wherein different cells of the screening library express a different randomized peptide antigen; and identifying a peptide sequence for a plurality of the randomized peptide antigens of the screening library that bind to the TCR or other macromolecule having one or more antigen binding domains. The method may further include expanding cells of the screening library having randomized peptide antigen that binds with the TCR or other macromolecule.

In certain aspects, the method includes embedding the sequences of the randomized peptide antigens that bind to the TCR or other macromolecule onto a latent space defined by molecular interactions of peptide antigens and antigen binding domains.

The method may also include a step of identifying clusters of mapped randomized peptide antigen sequences and performing a probability position matrix on the sequences of each cluster. In certain aspects, the method includes embedding the peptide activation predictions onto the latent space and identifying areas of the latent space proximal to peptide activation predictions for known TCR activators. A plurality of embedded peptide sequences may be identified in an area of the latent space proximal to the peptide activation prediction for one or more known TCR activators.

In certain aspects, the step of conducting one or more analyses using the peptide binding predictions and the peptide activation predictions to obtain a cross-reactivity profile includes identifying embedded peptide sequences of the randomized peptide antigens in an area of the latent space proximal to the peptide activation predictions for known TCR activators.

The present invention provides methods for treating a subject having a cancer that comprises cancer cells that display a target peptide. An exemplary method includes providing to the subject a therapeutic TCR that binds the target peptide to thereby treat the subject. In certain methods, the therapeutic TCR is identified by a process comprising: conducting one or more analyses to obtain a plurality of peptide binding predictions that are representative of the target peptide to which the TCR binds; conducting one or more analyses using the peptide binding predictions to obtain a plurality of peptide activation predictions that are representative of the identified target peptides that are predicted to activate the TCR; conducting one or more analyses using the peptide binding predictions and the peptide activation predictions to obtain a cross-reactivity profile of the TCR to thereby obtain a profile of the TCR; and identifying the therapeutic TCR using the cross-reactivity profile of the TCR with respect peptides predicted to activate the TCR.

In certain aspects, the step of conducting one or more analyses to obtain a plurality of peptide binding predictions comprises: contacting a screening library of cells with a TCR or other macromolecule having one or more antigen binding domains, wherein different cells of the screening library express a different randomized peptide antigen; and identifying a peptide sequence for a plurality of the randomized peptide antigens of the screening library that bind to the TCR or other macromolecule having one or more antigen binding domains.

In certain aspects, the method further includes expanding cells of the screening library having randomized peptide antigen that binds with the TCR or other macromolecule.

The methods of the invention may also include embedding the sequences of the randomized peptide antigens that bind to the TCR or other macromolecule onto a latent space defined by molecular interactions of peptide antigens and antigen binding domains. The method may also include identifying clusters of mapped randomized peptide antigen sequences and performing a probability position matrix on the sequences of each cluster. The method may also include identifying mapped randomized peptide antigen sequences that have a peptide sequence similar or identical to a peptide sequence of the target peptide.

In certain aspects, the method includes embedding the peptide activation predictions onto the latent space and identifying areas of the latent space proximal to peptide activation predictions for known TCR activators. The method may further include identifying a plurality of embedded peptide sequences in an area of the latent space proximal to the peptide activation prediction for one or more known TCR activators. In certain aspects, the step of conducting one or more analyses using the peptide binding predictions and the peptide activation predictions to obtain a cross-reactivity profile includes identifying embedded peptide sequences of the randomized peptide antigens in an area of the latent space proximal to the peptide activation predictions for known TCR activators.

The present invention also provides methods for preparing a composition comprising a TCR useful for treating cancer in a subject. An exemplary method may include obtaining tumor tissue from a subject and extracting tumor infiltrating lymphocytes from the tumor tissue; conducting one or more analyses to obtain a plurality of peptide binding predictions that are representative of peptide-HLA targets in the tumor tissue that bind to a TCR of an extracted tumor infiltrating lymphocyte; conducting one or more analyses using the peptide binding predictions to obtain a plurality of peptide activation predictions that are representative of identified target peptides that are predicted to activate the TCR; conducting one or more analyses using the peptide binding predictions and the peptide activation predictions to obtain a cross-reactivity profile of the TCR with respect to the target peptides identified to activate the TCR to thereby obtain a profile of the TCR; and causing engineered immune cells to express the TCR.

In certain aspects, the step of conducting one or more analyses to obtain a plurality of peptide binding predictions includes: contacting a screening library of cells with the TCR, wherein different cells of the screening library express a different peptide antigen, which has a peptide sequence of a neoantigen, a wildtype peptide, a spliced peptide, a human endogenous retrovirus (hERV), aeTSAs (aberrantly expressed, tumor specific antigens), a frameshift mutation, a gene fusion, an alternative splicing mutation, an aberrant translation, and/or an alternative promoter sequence expressed by a cell from the tumor tissue; and identifying a peptide sequence for a plurality of the different peptide antigens of the screening library that bind to the TCR.

In certain aspects, the method further includes expanding cells of the screening library having a peptide antigen that binds with the TCR.

The method may also include embedding the sequences of the randomized peptide antigens that bind to the TCR onto a latent space defined by molecular interactions of the peptide antigens and an antigen binding domain of the TCR. The method may also include identifying clusters of mapped peptide antigen sequences and performing a probability position matrix on the sequences of each cluster. In certain aspects, the method includes embedding the peptide activation predictions onto the latent space and identifying areas of the latent space proximal to peptide activation predictions for known TCR activators. The method may also include identifying a plurality of embedded peptide sequences in an area of the latent space proximal to the peptide activation prediction for one or more known TCR activators. In certain aspects, the step of conducting one or more analyses using the peptide binding predictions and the peptide activation predictions to obtain a cross-reactivity profile comprises identifying embedded peptide sequences of the different peptide antigens in an area of the latent space proximal to the peptide activation predictions for known TCR activators.

### BRIEF DESCRIPTION OF THE DRAWINGS

FIG. 1 provides a schematic of the methods and systems of the invention.

FIG. 2 provides a schematic of a yeast-display library used in module 1.

FIG. 3 provides a schematic of a yeast-display library used in module 1.

FIG. 4 shows an exemplary module 1 workflow.

FIG. 5 shows mCODE clustering and c-PPM fed into a conspred algorithm.

FIG. 6 shows an exemplary workflow for module 1.

FIG. 7 shows an exemplary workflow for module 2.

FIG. 8 shows a portion of a workflow for module 2.

FIG. 9 shows a portion of a workflow for module 2.

FIG. 10 shows latent space embedding for module 3.

FIG. 11 shows latent space embedding for module 3.

FIG. 12 shows latent space embedding for module 3.

FIG. 13 shows latent space embedding for module 3.

FIG. 14 shows an exemplary workflow using all three modules.

FIG. 15 shows the ability of the disclosed systems and methods to profile TCR activation.

FIG. 16 shows the continued improvement of the systems and methods of the disclosure.

FIG. 17 shows the continued improvement of the systems and methods of the disclosure.

### DETAILED DESCRIPTION

The present invention provides methods and systems that may identify potential antigens that bind to a particular T cell receptor (TCR), determine the immunogenicity of the potential antigens to activate the TCR, and exhaustively profile the receptor in terms of on-target and off-target reactivity to the potential antigens. As a result, the methods and systems of the disclosure are able to identify the specificity of a TCR, including “orphan” TCRs (TCRs of unknown antigen specificity), for particular antigens. The systems and methods are also able to predict efficacy and safety profile of immune cells activated by particular antigens.

### I. Definitions

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein is to be understood to include any integer within the recited range, unless otherwise indicated. As used herein, the term “about” means±20% of the indicated range, value, or structure, unless otherwise indicated. It should be understood that the terms “a” and “an” as used herein refer to “one or more” of the enumerated regions. Words using the singular or plural number also include the plural or singular number, respectively. Use of the word “or” in reference to a list of two or more items covers all of the following interpretations of the word: any of the items in the list, all of the items in the list, and any combination of the items in the list. Furthermore, the phrase “at least one of A, B, and C, etc.” is intended in the sense that one having skill in the art would understand the convention (e.g., “a system having at least one of A, B, and C” would include, but not be limited to, systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense that one having skill in the art would understand the convention (e.g., “a system having at least one of A, B, or C” would include, but not be limited to, systems that have A alone, B alone, C alone, A and B together, A and C together, B and C together, and/or A, B, and C together, etc.). As used herein, the terms “include,” “have,” and “comprise” are used synonymously, which terms and variants thereof are intended to be construed as non-limiting. Further, headings provided herein are for convenience only and do not interpret the scope or meaning of the claimed embodiments.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

The terms “treat,” “treating,” and “treatment” as used herein with regard to solid cancers refers to partial or total inhibition of tumor growth, reduction of tumor size, complete or partial tumor eradication, reduction or prevention of malignant growth, partial or total eradication of cancer cells, or some combination thereof. The terms “patient” and “subject” are used interchangeably herein.

A “subject in need thereof” as used herein refers to a mammalian subject, preferably a human, who has been diagnosed with cancer, is suspected of having cancer, and/or exhibits one or more symptoms associated with cancer.

The term “major histocompatibility complex” (MHC) proteins (also called human leukocyte antigens, HLA, or the H2 locus in the mouse) are protein molecules expressed on the surface of cells that confer a unique antigenic identity to these cells. MHC/HLA antigens are target molecules that are recognized by T-cells and natural killer (NK) cells as being derived from the same source of hematopoietic reconstituting stem cells as the immune effector cells (“self”) or as being derived from another source of hematopoietic reconstituting cells (“non-self”). Two main classes of HLA antigens are recognized: HLA class I and HLA class II. MHC proteins as used herein includes MHC proteins from any mammalian or avian species, e.g. primate sp., particularly humans; rodents, including mice, rats and hamsters; rabbits; equines, bovines, canines, felines, etc. Of particular interest are the human HLA proteins, and the murine H-2 proteins. Included in the HLA proteins are the class II subunits HLA-DPα, HLA-DPβ, HLA-DQα, HLA-DQβ, HLA-DRα and HLA-DRβ, and the class I proteins HLA-A, HLA-B, HLA-C, and β2-microglobulin. Included in the murine H-2 subunits are the class I H-2K, H-2D, H-2L, and the class II I-Aα, I-Aβ, I-Eα and I-Eβ, and β2-microglobulin.

As used herein, the term “class II HLA/MHC” binding domains comprise the α1 and α2 domains for the α chain, and the β1 and β2 domains for the β chain. Not more than about 10, usually not more than about 5, preferably none of the amino acids of the transmembrane domain will be included. The deletion will be such that it does not interfere with the ability of the α2 or β2 domain to bind target peptides (i.e., peptide ligands). Class II HLA/MHC binding domains also refers to the binding domains of a major histocompatibility complex protein that are soluble domains of Class II α and β chain. Class II HLA/MHC binding domains include domains that have been subjected to mutagenesis and selected for amino acid changes that enhance the solubility of the single chain polypeptide, without altering the peptide binding contacts.

As used herein, the term “class I HLA/MHC” binding domains includes the α1, α2 and α3 domain of a Class I allele, including without limitation HLA-A, HLA-B, HLA-C, H-2K, H-2D, H-2L, which are combined with β2-microglobulin. Not more than about 10, usually not more than about 5, preferably none of the amino acids of the transmembrane domain will be included. The deletion will be such that it does not interfere with the ability of the domains to bind target peptides (i.e., peptide ligands).

The “MHC binding domains”, as used herein, refers to a soluble form of the normally membrane-bound protein. The soluble form is derived from the native form by deletion of the transmembrane domain. The MHC binding domain protein is truncated, removing both the cytoplasmic and transmembrane domains and includes soluble domains of Class II alpha and beta chain. “MHC binding domains” also refers to binding domains that have been subjected to mutagenesis and selected for amino acid changes that enhance the solubility of the single chain polypeptide, without altering the peptide binding contacts.

“MHC context” as used herein refers to an interaction being in the presence of an MHC with non-covalent interactions with the MHC and an antigen. The function of MHC molecules is to bind peptide fragments derived from pathogens and display them on the cell surface for recognition by the appropriate T cells. Thus, TCR recognition can be influenced by the MHC protein that is presenting the antigen. The term MHC context refers to the recognition by a TCR of a given peptide when it is presented by a specific MHC protein.

An “allele” is one of the different nucleic acid sequences of a gene at a particular locus on a chromosome. One or more genetic differences can constitute an allele. An important aspect of the HLA gene system is its polymorphism. Each gene, MHC class I (A, B and C) and MHC class II (DP, DQ and DR) exists in different alleles. Current nomenclature for HLA alleles designated the alleles by numbers, as described by Marsh et al., “Nomenclature for factors of the HLA system”, 2010, Tissue Antigens 75:291-455, herein specifically incorporated by reference. For HLA protein and nucleic acid sequences, see Robinson et al. (2011), “The IMGT/HLA database”, Nucleic Acids Research 39 Suppl 1:D11171-6, herein specifically incorporated by reference.

“T cell receptor” (TCR), refers to an antigen/MHC binding heterodimeric protein product of a vertebrate (e.g., mammalian, TCR gene complex, including the human TCR α, β, γ, and δ chains). For example, the complete sequence of the human β TCR locus has been sequenced, as published by Rowen 1996; the human TCR locus has been sequenced and re-sequenced, for example, see Mackelprang 2006; see a general analysis of the T-cell receptor variable gene segment families in Arden 1995; each of which is herein specifically incorporated by reference for the sequence information provided and referenced in the publication.

T cells are a part of the adaptive immune system that protects against pathogens and cancer. T cells act through extracellular recognition of an antigen target by the TCR, which is specific for short peptides presented on the human leukocyte antigen (HLA) on cells (Birnbaum et al., (2014) Cell 157, 1073-1087). Each T cell may express a unique T cell receptor (TCR), which has the ability to specifically bind to major histocompatibility complex (MHC) molecules presenting peptides. TCR recognition of peptide-MHC (pMHC) drives T cell development, survival, activation and effector functions. Though TCR ligands have relatively low affinities for their cognate TCR(s) (1-100 λM), the TCRs are remarkably sensitive—they may require as few as 10 agonist peptides to fully activate a T cell. After recognition, a signaling cascade allows T cells to carry out their immune functions.

Structural studies investigating TCR recognition of pMHC have revealed that the vast majority of TCR-pMHC complexes share a consistent binding orientation, driven by conserved contacts between the tops of the MHC helices and the germline-encoded TCR CDR1 and CDR2 loops. Alteration to the typical TCR-pMHC interaction is correlated with reduced or stopped signaling.

TCRs have shown an ability to balance cross-reactivity with specificity. The total number of T cells necessary to uniquely recognize every possible pMHC combination is extremely high, and since there are few if any gaps characterized in the TCR repertoire, it has been suggested that TCR cross-reactivity is a requirement of functional antigen recognition.

Numerous strategies have been employed to determine the specificity of “orphan” TCRs (Birnbaum et al., (2012)Immunol Rev 250, 82-101). For example, mass spectrometry may provide an unbiased method of antigen isolation, but is restricted to experiment requiring large cell numbers, typically, and the targets must still be presented by a correct HLA. Most investigations of T cell antigen specificities involve testing candidate antigens empirically. Studies of anti-tumor T cell specificities have revealed productive T cell responses towards neo-antigens. These studies generally involve sequencing tumors to identify mutations, using an epitope prediction algorithm to predict immunogenic mutant peptides, and testing for T cell responses directed at these mutant peptides (Kreiter et al., (2015) Nature 520, 692-696; Rajasagi et al., (2014) Blood 124, 453-462; Tran et al., (2014) Science 344, 641-645). Other strategies query established T cell specificities in patients by using pHLA multimers (Bentzen et al., (2016) Nat Biotechnol 34, 1037-1045; Newell et al., (2013) Nat Biotechnol 31, 623-629).

The terms “recipient,” “individual,” “subject,” “host,” and “patient” are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

The terms “peptide,” “polypeptide,” and “protein” are used interchangeably to refer to a polymer of amino acid residues, and are not limited to a minimum length, though a number of amino acid residues may be specified (e.g., 9mer is nine amino acid residues). Polypeptides may include amino acid residues including natural and/or non-natural amino acid residues. Polypeptides may also include fusion proteins. The terms also include post-expression modifications of the polypeptide, for example, glycosylation, sialylation, acetylation, phosphorylation, and the like. In some embodiments, the polypeptides may contain modifications with respect to a native or natural sequence, as long as the protein maintains the desired activity. These modifications may be deliberate, such as through site-directed mutagenesis, or may be accidental, such as through mutations of hosts which produce the proteins or errors due to PCR amplification.

The term “acidic residue” refers to amino acid residues in D- or L-form having sidechains comprising acidic groups. Exemplary acidic residues include D and E.

The term “amide residue” refers to amino acids in D- or L-form having sidechains comprising amide derivatives of acidic groups. Exemplary residues include N and Q.

The term “aromatic residue” refers to amino acid residues in D- or L-form having sidechains comprising aromatic groups. Exemplary aromatic residues include F, Y, and W.

The term “basic residue” refers to amino acid residues in D- or L-form having sidechains comprising basic groups. Exemplary basic residues include H, K, and R.

The term “hydrophilic residue” refers to amino acid residues in D- or L-form having sidechains comprising polar groups. Exemplary hydrophilic residues include C, S, T, N, and Q.

The term “nonfunctional residue” refers to amino acid residues in D- or L-form having sidechains that lack acidic, basic, or aromatic groups. Exemplary nonfunctional amino acid residues include M, G, A, V, I, L, and norleucine (Nle).

The term “neutral hydrophobic residue” refers to amino acid residues in D- or L-form having sidechains that lack basic, acidic, or polar groups. Exemplary neutral hydrophobic amino acid residues include A, V, L, I, P, W, M, and F.

The term “polar hydrophobic residue” refers to amino acid residues in D- or L-form having sidechains comprising polar groups. Exemplary polar hydrophobic amino acid residues include T, G, S, Y, C, Q, and N.

The term “hydrophobic residue” refers to amino acid residues in D- or L-form having sidechains that lack basic or acidic groups. Exemplary hydrophobic amino acid residues include A, V, L, I, P, W, M, F, T, G, S, Y, C, Q, and N.

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally, or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include sulfur-containing: Met and Cysteine (Cys or C); acidic: Asp, Glu, Asn, and Gln; small aliphatic, nonpolar, or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W. H. Freeman and Company. Variant proteins, peptides, polypeptides, and amino acid sequences of the present disclosure can, in certain embodiments, comprise one or more conservative substitutions relative to a reference amino acid sequence.

“Nucleic acid molecule” or “polynucleotide” refers to a polymeric compound including covalently linked nucleotides comprising natural subunits (e.g., purine or pyrimidine bases). Purine bases include adenine and guanine, and pyrimidine bases include uracil, thymine, and cytosine. Nucleic acid molecules include polyribonucleic acid (RNA) and polydeoxyribonucleic acid (DNA), which includes cDNA, genomic DNA, and synthetic DNA, either of which may be single or double-stranded. A nucleic acid molecule encoding an amino acid sequence includes all nucleotide sequences that encode the same amino acid sequence.

“Percent (%) sequence identity” with respect to a reference polypeptide sequence is the percentage of amino acid residues in a candidate sequence that is identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known, for instance, using publicly available computer software such as BLAST, BLAST-2, ALIGN, or Megalign (DNASTAR) software, or other software appropriate for nucleic acid sequences. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a some % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “isolated” means that the material is removed from its original environment (e.g., the natural environment if it is naturally occurring). Such nucleic acid could be part of a vector and/or such nucleic acid or polypeptide could be part of a composition (e.g., a cell lysate), and still be isolated in that such vector or composition is not part of the natural environment for the nucleic acid or polypeptide.

As used herein, the terms “homologous,” “homology,” or “percent homology” when used herein to describe a nucleic acid sequence relative to a reference sequence, can be determined using the formula described by Karlin & Altschul 1990, modified as in Karlin & Altschul 1993. Such a formula is incorporated into the basic local alignment search tool (BLAST) programs of Altschul 1990. Percent homology of sequences can be determined using the most recent version of BLAST, as of the filing date of this application. Homologous sequences described herein include sequences having the same percentage identity as the indicated percentage homology. Sequences sharing a percentage identity are understood in the art to mean those sequences sharing the indicated percentage of same residues over the length of the reference sequence (e.g., the linker or leader sequences disclosed herein and in the sequence listing).

A “functional variant” refers to a polypeptide or polynucleotide that is structurally similar or substantially structurally similar to a parent or reference compound of this disclosure, but differs, in some contexts slightly, in composition (e.g., one base, atom, or functional group is different, added, or removed; or one or more amino acids are substituted, mutated, inserted, or deleted), such that the polypeptide or encoded polypeptide is capable of performing at least one function of the encoded parent polypeptide with at least 50% efficiency of activity of the parent polypeptide.

As used herein, a “functional portion” or “functional fragment” refers to a polypeptide or polynucleotide that comprises only a domain, motif, portion, or fragment of a parent or reference compound, and the polypeptide or encoded polypeptide retains at least 50% activity associated with the domain, portion, or fragment of the parent or reference compound.

In certain embodiments, a functional variant or functional portion or functional fragment each refers to a “signaling portion” of an effector molecule, effector domain, costimulatory molecule, or costimulatory domain. In other aspects, a functional variant or functional portion or functional fragment each refers to a linking function or a leader peptide function as disclosed herein. In certain aspects, a functional variant/portion/fragment refers to a linking function or a leader peptide function as described herein. In specific aspects, variant linkers and leader peptides are at least 60% as efficient, at least 70% as efficient, at least 80% as efficient, at least 90% as efficient, at least 95% as efficient, or at least 99% as efficient as the reference/parent polypeptides disclosed herein.

The term “expression,” as used herein, refers to the process by which a polypeptide is produced based on the encoding sequence of a nucleic acid molecule, such as a gene. The process may include transcription, post-transcriptional control, post-transcriptional modification, translation, post-translational control, post-translational modification, or any combination thereof. An expressed nucleic acid molecule is typically operably linked to an expression control sequence (e.g., a promoter).

The term “operably linked” refers to the association of two or more nucleic acid molecules on a single nucleic acid fragment so that the function of one is affected by the other.

As used herein, “expression vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. Here, “plasmid,” “expression plasmid,” “virus,” and “vector” are often used interchangeably.

The terms “modify,” “modifying,” or “modification” in the context of making alterations to nucleic compositions of a cell, and the term “introduced” in the context of inserting a nucleic acid molecule into a cell, include reference to the alteration or incorporation of a nucleic acid molecule in a eukaryotic cell wherein the nucleic acid molecule may be incorporated into the genome of a cell and converted into an autonomous replicon. “Modification” or “introduction” of nucleic compositions in a cell may be accomplished by a variety of methods known in the art, including, but not limited to, transfection, transformation, transduction, or gene editing. As used herein, the term “engineered,” “recombinant,” “modified,” or “non-natural” refers to an organism, microorganism, cell, nucleic acid molecule, or vector that includes at least one genetic alteration or has been modified by introduction of an exogenous nucleic acid molecule, wherein such alterations or modifications are introduced by genetic engineering. Genetic alterations include, for example, modifications and/or introductions of expressible nucleic acid molecules encoding polypeptide, such as additions, deletions, substitutions, mutations, or other functional changes of a cell's genetic material.

The term “construct” refers to any polynucleotide that contains a recombinant nucleic acid molecule. A construct may be present in a vector (e.g., a bacterial vector, a viral vector) or may be integrated into a genome. A “vector” is a nucleic acid molecule that is capable of transporting another nucleic acid molecule. Vectors may be, for example, plasmids, cosmids, viruses, an RNA vector or a linear or circular DNA or RNA molecule that may include chromosomal, non-chromosomal, semi-synthetic, or synthetic nucleic acid molecules. Exemplary vectors are those capable of autonomous replication (episomal vector), capable of delivering a polynucleotide to a cell genome (e.g., viral vector), or capable of expressing nucleic acid molecules to which they are linked (expression vectors).

As used herein, the term “host” refers to a cell or microorganism targeted for genetic modification with a heterologous nucleic acid molecule to produce a polypeptide of interest. In certain embodiments, a host cell may optionally already possess or be modified to include other genetic modifications that confer desired properties related, or unrelated to, biosynthesis of the heterologous protein.

As used herein, “enriched” or “depleted” with respect to amounts of cell types in a mixture refers to an increase in the number of the “enriched” type, a decrease in the number of the “depleted” cells, or both, in a mixture of cells resulting from one or more enriching or depleting processes or steps. In certain embodiments, amounts of a certain cell type in a mixture will be enriched and amounts of a different cell type will be depleted, such as enriching for CD4+ cells while depleting CD8+ cells, or enriching for CD8+ cells while depleting CD4+ cells, or combinations thereof.

“Antigen” as used herein refers to an immunogenic molecule that provokes an immune response. This immune response may involve antibody production, activation of specific immunologically-competent cells, or both. An antigen may be, for example, a peptide, glycopeptide, polypeptide, glycopolypeptide, polynucleotide, polysaccharide, lipid, or the like. It is readily apparent that an antigen can be synthesized, produced recombinantly, or derived from a biological sample. Exemplary biological samples that can contain one or more antigens include tissue samples, tumor samples, cells, biological fluids, or combinations thereof. Antigens can be produced by cells that have been modified or genetically engineered to express an antigen.

The term “epitope” includes any molecule, structure, amino acid sequence, or protein determinant that is recognized and specifically bound by a cognate binding molecule, such as a chimeric antigen receptor, or other binding molecule, domain, or protein.

“Exogenous” with respect to a nucleic acid or polynucleotide indicates that the nucleic acid is part of a recombinant nucleic acid construct or is not in its natural environment. For example, an exogenous nucleic acid can be a sequence from one species introduced into another species (i.e., a heterologous nucleic acid). Typically, such an exogenous nucleic acid is introduced into the other species via a recombinant nucleic acid construct. An exogenous nucleic acid also can be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, for example, non-native regulatory sequences flanking a native sequence in a recombinant nucleic acid construct. In addition, stably transformed exogenous nucleic acids typically are integrated at positions other than the position where the native sequence is found. The exogenous elements may be added to a construct, for example, using genetic recombination. Genetic recombination is the breaking and rejoining of DNA strands to form new molecules of DNA encoding a novel set of genetic information.

A “T cell” or “T lymphocyte” is an immune system cell that matures in the thymus and produces TCRs, including αβT cells and γδT cells. T cells can be naïve (not exposed to antigen; increased expression of CD62L, CCR7, CD28, CD3, CD127, and CD45RA, and decreased expression of CD45RO as compared to TCM), memory T cells (TM) (antigen-experienced and long-lived), and effector cells (antigen-experienced, cytotoxic). TM can be further divided into subsets of central memory T cells (TCM, increased expression of CD62L, CCR7, CD28, CD127, CD45RO, and CD95, and decreased expression of CD54RA as compared to naïve T cells) and effector memory T cells (TEM, decreased expression of CD62L, CCR7, CD28, CD45RA, and increased expression of CD127 as compared to naïve T cells or TCM).

The term “leader sequence,” used interchangeably with “signal sequence” and also referred to as “leader peptide” or “signal peptide” herein, is an amino acid sequence at the N-terminus of a peptide or a polypeptide that confers a trafficking preference to the peptide or the polypeptide, directs the nascent peptide or polypeptide to the ER, facilitates ER to Golgi transport, and/or facilitates aspects of late secretory processing. The term “leader sequence” also refers to a nucleotide sequence encoding the leader peptide.

In addition, it should be understood that the individual constructs, or groups of constructs, derived from the various combinations of the structures and subunits described herein, are disclosed by the present disclosure to the same extent as if each construct or group of constructs was set forth individually. Thus, selection of particular structures or particular subunits is within the scope of the present disclosure.

The terminology used in the description is intended to be interpreted in its broadest reasonable manner, even though it is being used in conjunction with a detailed description of identified embodiments.

### II. Systems and Methods Overview

The presently disclosed systems and methods have three main components or modules. Each of these components may include one or more wet lab steps and/or one or more computational steps, which preferably include a machine learning system.

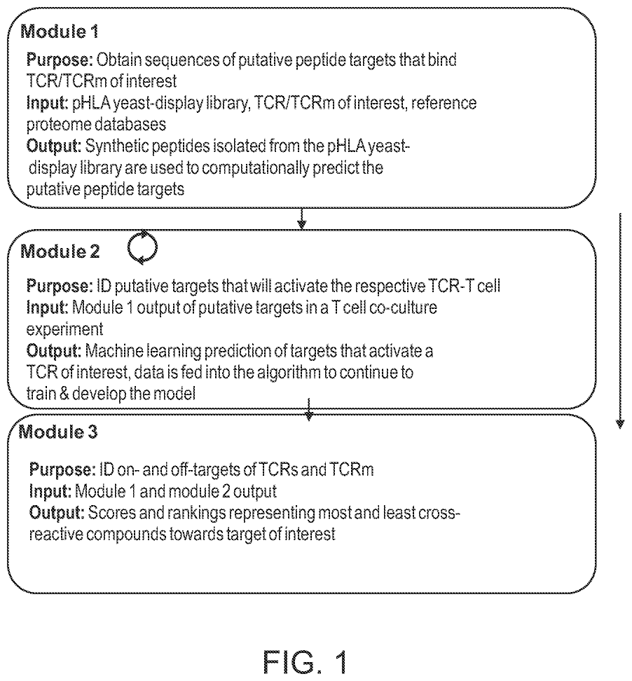

FIG. 1 provides an overview of the three modules of the systems and methods of the invention.

As shown, the first component or module deep sequencing data from pHLA yeast-display libraries to predict target antigens, e.g., cognate peptide-HLAs, for a particular TCR. This module can similarly predict target antigens of, for example, a TCR mimetic (TCRm). The identified peptide sequences may be used to generate a machine learning model that compares the peptide sequences with, for example, one or more proteome databases to predict potential endogenous ligands of the TCR. The first module may also use the peptide sequence to identify sequence or binding motifs in the peptide sequences, which may identify superbinders—peptide sequences with a higher affinity for a TCR than endogenous antigen.

The second module determines the immunogenicity of peptides to activate a particular TCR of interest, e.g., an orphan TCR. The second module uses T-cell/TCR activation data to train a machine learning model. Once trained, the model may be used to predict the activation potential for peptides or antigens identified by module 1.

The third module is used to exhaustively profile a TCR of interest in terms of on-target and off-target cross reactivity. This module may include constructing an activation neighborhood in a latent space, which may be continually updated to reveal new activators until the cross-reactivity profile of the TCR is determined based on an absence of any new activators.

The three modules leverage the pHLA yeast-display libraries to determine TCR specificity encoded in the small recognition kernel of the MHC-bound peptide visible to the TCR. Sequence data from the yeast-display libraries is sufficient to enable reconstruction of entire sequences of endogenous peptides to TCRs of unknown specificity. This has important implications for the identification of antigens in T cell mediated diseases. T cells provide an avenue of therapeutic treatment in infectious diseases, autoimmunity, allergy and cancer. However, most TCRs have little known information about their specificities, especially in humans, due to the limitations of prior methods.

The present invention combines TCR analysis methods with a refined version of the yeast display library screening approach to discover novel pHLA specificities TCRs. This has broad implications for our understanding of T cell specificities in cancer and can be applied to other diseases.

To our knowledge, this is the first instance of TCR ligand identification using a combinatorial biology screening technology, in which three TCRs were found to be specific for wildtype antigens, which have roles in cancer. The presently disclosed systems and methods are able to identify multiple mimotope peptides that stimulate a TCR of interest, often more potently than the native peptide. Akin to neoantigens, the synthetic peptide antigens or mimotopes have utility as DNA, RNA or peptide vaccines to stimulate particular antigen-specific T cells and generate a more immunogenic response than the self-antigen that the immune response is likely tolerant towards.

The success of predicting the cognate tumor antigen from deep sequencing selection data depends on the high efficacy of the machine learning models used in one or more of modules 1, 2, or 3, as described herein. Using the presently disclosed systems and methods, large numbers of TCRs from a given tumor may be screened, increasing the odds of linking selection data to the cognate antigen, especially when coupled to relevant patient data including RNA expression and/or mass spectrometry of eluted peptides.

The systems and methods of the invention may find broad applications. Two principal applications are available for this method in immunotherapy: 1) to identify endogenous and mimotope ligands for orphan TCRs and/or 2) as a means of classifying TCRs based on peptide antigen specificities, which will allow the identification of clinical candidate TCRs that recognize shared antigens across patients. Shared TCRs can either be receptors that share similar TCR sequence, which can potentially lead to shared antigen specificity, or TCRs that do not have any shared sequence but recognize the same antigen. Such TCRs recognizing shared antigens would be especially useful in engineered T cell or vaccine therapies. As TCR sequencing continues to advance and more TCR sequencing data becomes available, the presently disclosed systems and methods may be used infer TCR restriction for patient HLA and infer a common TCR specificity for convergent TCR sequence clusters. This enables TCR ligand identification to be more effectively directed at impactful TCRs with known HLA restriction.

III. Module 1—Identifying Potential Ligands that Bind TCR/TCRm of Interest

The general purpose of Module 1 of the systems and methods of the invention is to predict targets/antigens to which a TCR/TCRm of interest, such as an “orphan” TCR isolated from a tumor infiltrating lymphocyte (TIL). Module 1 uses the peptides identified in the yeast-display selections generate a recognition landscape of sequences for each TCR.

In certain aspects, module 1 includes the use of pHLA yeast-display libraries to provide sequence data to predict potential antigens of a TCR/TCRm of interest.

Yeast-display libraries may be prepared and used in, for example, as provided in PCT international application publications WO 2015/153969, WO 2018/175585, WO 2020/047502, and WO 2021/168388, each of which is incorporated herein by reference.

In certain aspects, a library of single chain polypeptides is generated. Each polypeptide may include the binding domains of a major histocompatibility complex (MHC) protein and diverse peptide ligands. The library is initially generated as a population of polynucleotides encoding the single chain polypeptide operably linked to an expression vector, which library may comprise at least 10⁶, at least 10⁷, or as is most common, at least 10⁸ different peptide ligand coding sequences, and may contain up to about 10¹³, 10¹⁴ or more different ligand sequences. Polypeptides from the library are introduced into suitable host cells, which in turn expresses the encoded polypeptide. Preferably, the host cells are yeast cells. The number of unique host cells expressing the polypeptide is generally less than the total predicted diversity of polynucleotides, e.g., up to about 5×10⁹ different specificities, up to about 10⁹, up to about 5×10⁸, up to about 10⁸, etc.

Preferably, the peptide ligand is from about 8 to about 20 amino acids in length, usually from about 8 to about 18 amino acids, from about 8 to about 16 amino acids, from about 8 to about 14 amino acids, from about 8 to about 12 amino acids, from about 10 to about 14 amino acids, from about 10 to about 12 amino acids. As a fully random library may represent a large number of possible combinations, in preferred aspects, the peptide ligand diversity is limited at the residues that anchor the peptide to the MHC binding domains, which are referred to herein as MHC anchor residues. The position of the anchor residues in the peptide may be determined by specific MHC binding domains. For example, class I binding domains have anchor residues at the P2 position, and at the last contact residue. Class II binding domains have an anchor residue at P1, and depending on the allele, at one of P4, P6 or P9.

A peptide may be provided as short antigenic sequence active in stimulating T cells; or may be provided in the form of the larger protein, e.g., an intact domain, a soluble protein portion, a complete protein, etc. In certain aspects, peptide antigens are identified that are shared between patients and provide a means for broadly applicable therapy. In other aspects identification of antigens provides for a personalized medicine approach

Each yeast displays a unique ligand peptide that is genetically encoded. A typical library contains ˜10⁸ to 10⁹ unique peptides, which are selected by a TCR of interest. The libraries have theoretical nucleotide diversities dictated by the peptide length and library composition. The functional diversity represents the true capacity of the physical libraries based on yeast colony counting after limiting dilution of the library. The displayed peptides may be fragments of naturally occurring antigenic proteins, may be fragments of neoantigenic proteins that are the subject of somatic mutation during tumorigenesis, and/or may be a synthetically generated mimic of an antigenic protein. The synthetic peptides can act as highly potent agonists of T cell receptors. A peptide may be provided as short antigenic sequence active in stimulating T cells; or may be provided in the form of the larger protein, e.g., an intact domain, a soluble protein portion, a complete protein, etc. In some embodiments, peptide antigens are identified that are shared between patients and provide a means for broadly applicable therapy. In other embodiments identification of antigens provides for a personalized medicine approach.

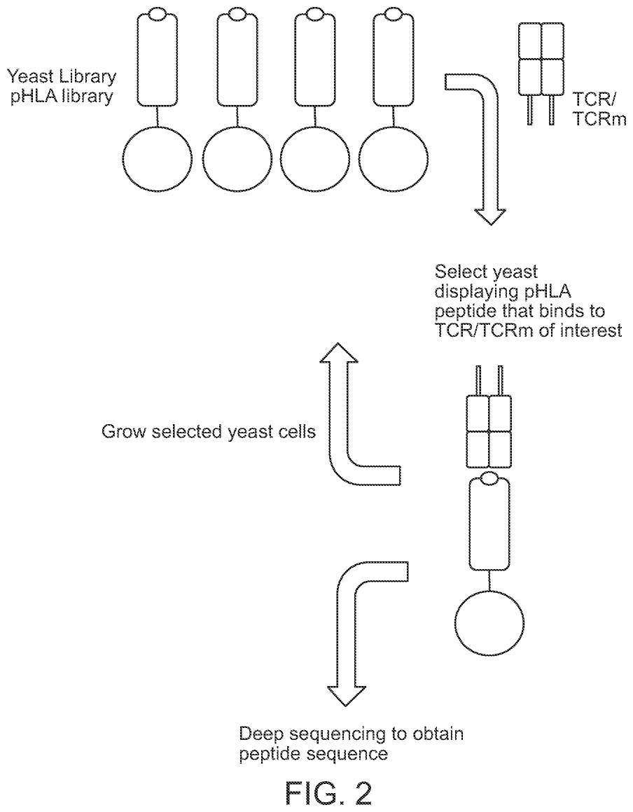

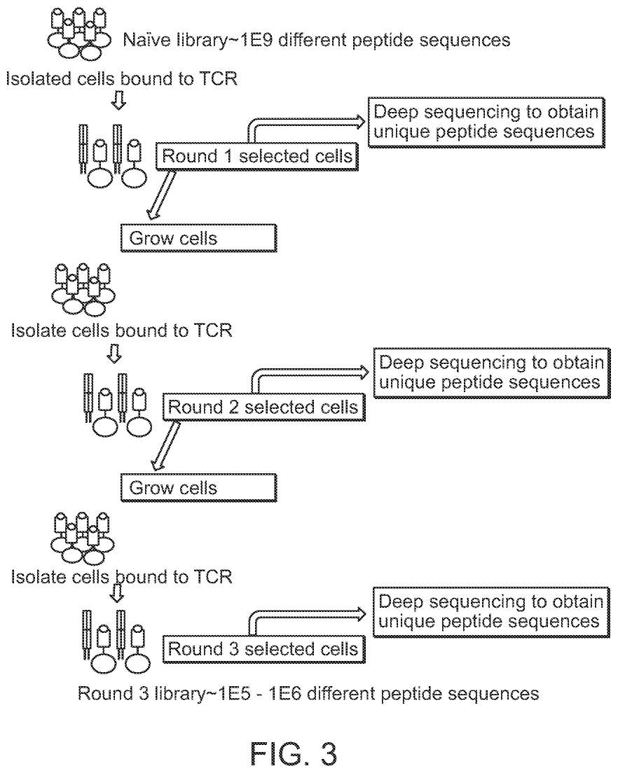

FIGS. 2 and 3 provide schematic overviews of the use of pHLA yeast-display libraries in the methods and systems of the invention.

As shown, a pHLA yeast-display library is prepared, in which yeast cells express different introduced protein ligands. A TCR of interest, such as an orphan TCR from a TIC, is introduced to the yeast-display library cells. In certain aspects, the TCR of interest is multimerized to enhance binding, and used to select for host cells expressing those introduced protein ligands that bind to the TCR of interest. Iterative rounds of selection are performed, i.e., the cells that are selected in the first round provide the starting population for the second round. Usually at least three and more usually at least four rounds of selection are performed.

Yeast may be enriched, for example, using an affinity-based selection using a bead-multimerized TCR and grown for iterative rounds of selection. This causes the ligand peptides with high affinity for the TCR to be successively enriched across the rounds of selection, and all yeast DNA is deep-sequenced.

In certain aspects, these synthetic peptide sequences are used to generate a model to make predictions for TCR ligands derived from the human proteome and/or patient-specific exome.

In certain aspects, the yeast-display selection is used to de-orphanize a TCR of unknown antigen specificity. The peptides selected by a TCR from the yeast-display selection generate a recognition landscape for a particular TCR, which is then used to make predictions of antigen specificity for orphan TCRs. Predicted targets can be validated, for example, using in a T cell stimulation assay or via a trained machine learning model.

In certain aspects, the present invention provides a method of determining the set of polypeptide ligands that bind to a T cell receptor of interest, comprising the steps of: performing multiple rounds of selection of a polypeptide library as set forth herein with a T cell receptor of interest; performing deep sequencing of the peptide ligands that are selected; inputting the sequence data to computer readable medium, where it is used to generate a search algorithm embodied as a program of instructions executable by computer and performed by means of software components loaded into the computer.

Thus, the present invention also provides software products tangibly embodied in a machine-readable medium. The software product may include instructions operable to cause one or more data processing apparatus to perform operations comprising: obtaining peptide sequences from a yeast-display library after each round of selection; clustering the peptide sequence reads after each round; producing a cluster specific probability position matrix for each cluster; obtain one or more ligand consensus sequence. The probability position matrix for each cluster may be used to compare the yeast-display peptide ligands with one or more reference proteome databases. For example, the proteome databases may include one or more of peptide sequences for neoantigens, wildtype peptides, spliced peptides, human endogenous retroviruses (hERV), aeTSAs (aberrantly expressed TSAs), frameshift mutations, gene fusions, alternative splicing mutations, aberrant translations, and/or an alternative promoter sequence expressed by a cell from the tumor tissue.

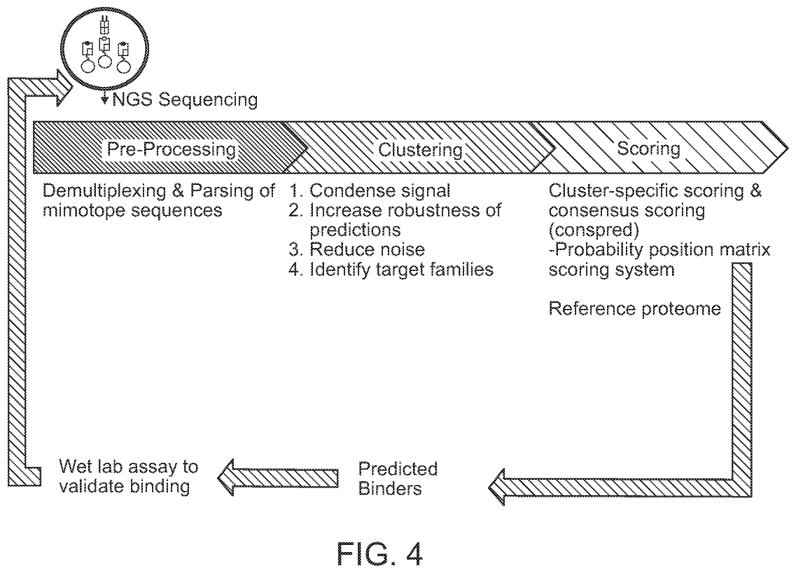

FIG. 4 provides an overview of an exemplary analysis pipeline in module 1. As shown, nucleic acid sequences from the yeast-display library are obtained after each round of selection via next generation deep sequencing and provided in a FASTQ format. The analysis pipeline includes pre-processing, which may include, for example, demultiplexing and parsing the sequence reads to identify the peptide sequences of the displayed peptides (mimotopes). The sequences of peptides may be determined in module 1 by any convenient methods of high throughput sequencing. In certain aspects, the peptide ligands include ligands of varying sizes. Thus, in certain methods of the invention, peptides may be parsed based on peptide length. In certain aspects, module 1 incorporates deconvolution of the peptide sequence library to account for peptides of varying length.

The resulting Sequences may be analyzed, for example, by using clustering algorithms and/or methods. In preferred aspects, the clustering method/algorithm is minimal common oncology data element (mCODE). In certain aspects, clustering the reads includes calculating the reverse hamming distance between all peptides for which sequences were obtained. Reverse hamming distances are hamming distances subtracted from the total length of a peptide and represent the number of shared amino acids between two peptides. They may be calculated using Matlab (Mathworks Inc.) by iterating through each peptide against all other peptides selected during a round. The output score generated is the number of matching amino acid positions between peptides. Based on the reverse hamming distances, peptides may be clustered using mCODE/Cytoscape.

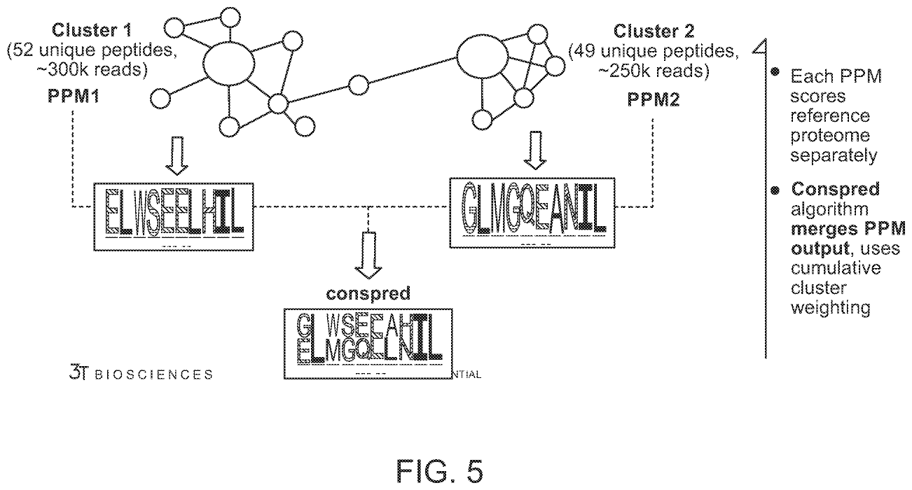

FIG. 5 provide an exemplary schematic of clustering peptide sequences according to the invention. As shown in FIG. 5, after each round of selection, the identified yeast-display library peptide sequences are clustered using mCODE. The mCODE clustering condenses the signal, increases the robustness of potential ligand predictions, and reduces the noise relative to other clustering modalities. mCODE uses vertex weighting and weighs nodes based on local neighborhood density. Nodes with high weights are selected as seed nodes of initial clusters. The clusters are augmented by outwardly traversing from the seeds.

As further shown in FIG. 5, cluster position probability matrices (c-PPM) are created using the peptides in each cluster. In certain aspects, generating the c-PPM for each cluster includes, scoring the yeast-display library peptides in each cluster using a PPM. The resulting PPM for each cluster may be fed to an algorithm (conspred algorithm) that merges the PPM outputs using cumulative cluster weighting. The resulting conspred sequences (predicted consensus sequence(s)) for the yeast-display peptides may be used to search one or more proteome databases (e.g., for human proteins (Uniprot) or patient-specific exomes) to score peptides of fixed lengths using a sliding window. Substitution matrices are made by determining the frequency of all amino acids per position of the peptide. A cutoff of 0.1% frequency for an amino acid at a given position may be instituted to remove noise.

In certain aspects, the c-PPM outputs may be used to produce substitution matrices from all rounds 3 of yeast-display library selection and used to search one or more proteome databases to score peptides of fixed lengths using a sliding window. Substitution matrices are made by determining the frequency of all amino acids in the display peptides. In certain aspects, the substitution matrices are made by determining the frequency of all amino acids per position of the peptide. The scores of the peptides may calculated as the product of amino acid frequencies at each position.

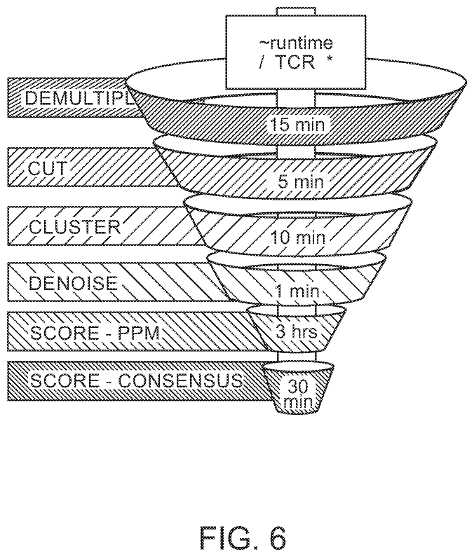

FIG. 6 provides an overview of the steps used in an exemplary version of module 1 as described herein.

In certain aspects, module 1 incorporates a machine learning model to compare the yeast-display library peptide sequences with one or more proteome databases.

Preferably, the machine learning model considers peptides as whole entities rather than taking each individual position of the peptide as independent of every other. Sequencing data including peptide sequences and round counts may be pre-processed in R to remove any peptide sequences that have fewer than a certain number of counts across all rounds. In certain aspects, the data is normalized by multiplying each round count by the average number of counts across the rounds and then divided by the number of counts in a given round. An adapted fitness score may be used to score each peptide in the library derived from a fitness function represented by an exponential curve fit to each peptide through the normalized round counts.

The model may be generated using the fitness scores for each peptide and the peptides represented as a 20×L matrix, where L is the length of the peptide sequence. The 20 rows of the matrix relate to the 20 possible amino acids. Amino acids are represented as a one-hot vector, in which a vector contains a single 1 with the remaining being 0s. The matrix representing the peptide may be flattened to a feature vector of length 20×L for use in training a neural network. The one-hot matrix may be used as input and the fitness scores used as output.

Preferably, the machine learning model models the competitive growth environment of the peptide sequences across all rounds of selection. This provides a >20-fold data augmentation when compared with prior methods. In certain aspects, module 1 employs transfer learning to train the machine learning model. Transfer learning involves training the machine learning model using one or more models that have already been trained to identify potential TCR ligands in a database that correlate to the selected yeast-display library peptide sequences. Preferably, the machine learning model is trained using one or more highly-trained long short-term memory (LSTM) and transformer embedding models. LSTM is an artificial recurrent neural network (RNN) architecture. Unlike feedforward neural networks, such as multi-layer perceptron neural networks, LSTM uses feedback connections. In feedback neural networks, connections may form cycles or loops such that learned information can move throughout layers of the network. In contrast, feedforward layers of the network do not form a cycle, and data can only move in the forward direction. This distinction means that feedforward neural networks are capable of capturing dynamic temporal information.

One potential drawback of feedback neural networks is that they can be complex and slow, as the process data in sequence. To overcome this disadvantage, transformer embedding can be used in conjunction with the LSTM model. Transformer embedding models are able to process sequential data even when it is not provided sequentially to the model. Thus, the transformer embedding allows the advantages of a feedback model to proceed efficiently.

In certain aspects, prior yeast-display library binding results are used to train a machine learning model. Assigning training data for a training data set may be random or not completely random. One or more criteria may be used during the assignment. Any suitable method may be used to assign the data to the training or testing data sets.

In certain aspects, module 1 includes a training module to train the machine learning module by extracting a feature set from the training data set 410 according to one or more feature selection techniques. The training module may train the machine learning model by extracting a feature set from the training data set that includes statistically significant features of positive examples (e.g., mimotopes that bind a TCR) and statistically significant features of negative examples. The training module may extract a feature set from the training data set in a variety of ways. The training module extract features multiple times. Subsequent rounds of feature extraction may use the same or different feature extraction techniques.

The training module may use the feature set(s) to build one or more machine learning-based classification models configured to identify potential antigen candidates for a TCR of interest by comparing the yeast-display peptide library sequences with peptide sequences in a proteome database.

After the training module generates a feature set(s), the training module may generate a machine learning-based classification model based on the feature set(s).

The extracted features may be combined in a classification model trained using a machine learning approach such as discriminant analysis; decision tree; a nearest neighbor (NN) algorithm (e.g., k-NN models, replicator NN models, etc.); statistical algorithm (e.g., Bayesian networks, etc.); clustering algorithm (e.g., k-means, mean-shift, etc.); neural networks (e.g., reservoir networks, artificial neural networks, etc.); support vector machines (SVMs); logistic regression algorithms; linear regression algorithms; Markov models or chains; principal component analysis (PCA) (e.g., for linear models); multi-layer perceptron (MLP) ANNs (e.g., for non-linear models); replicating reservoir networks (e.g., for non-linear models, typically for time series); random forest classification; a combination thereof and/or the like.

The candidate feature(s) and the machine learning module may predict potential endogenous or exogenous antigens for a TCR of interest. The result for each potential TCR antigen may include a confidence level that corresponds to a likelihood or a probability that the receptor sequence will bind to a peptide. The top performing candidate feature(s) may be used to predict whether a particular antigen will bind to a TCR of interest. For example, a potential peptide ligand sequence for a TCR of interest may be determined. The new TCR sequence may be provided to the machine learning module which may, based on the top performing candidate feature, classify the new potential peptide as either binding (yes) or not binding (no) to a TCR/TCRm of interest.

In certain aspects, the training module uses supervised, unsupervised, and/or semi-supervised (e.g., reinforcement based) machine learning-based classification models. In certain aspects, training is done in Lua with the Torch package.

In certain aspects, the resulting model is used to score given peptides from the proteome database (e.g., Uniprot) patient-specific exomes using peptides isolated from an L-length sliding window converted to one-hot matrices for neural network input. P-values and Bonferroni-corrected p-values were calculated for each peptide, representing the probability of randomly selecting, from the whole proteome, a peptide with fitness score as high as or higher than the scored peptide.

The analysis of the yeast-display peptides using module 1 may reveal that the selected set of peptide ligands exhibit a restricted choice of amino acids at residues, e.g., the residues that contact the TCR, which information can be input into a machine learning algorithm as described that can be used to analyze public databases for all peptides that meet the criteria for binding, and which provides a set of peptides that meet these criteria. The first module may also use the peptide sequence to identify sequence or binding motifs in the peptide sequences, which may identify superbinders—peptide sequences with a higher affinity for a TCR than endogenous antigen.

In certain aspects, module 1 incorporates one or more quality control and/or validation steps. Quality control steps may, for example, provide enhanced identification of biological versus artefactual TCR antigen specificity peptide groups. The quality control may be improved using machine learning trained on the historical outcome of screens, e.g., peptide predictions validated using wet lab assays and/or further iterations using module 1. Unlike prior methods that use exome data to identify patient-specific neoantigens that can serve as potential targets of the T cell immune response, the presently disclosed systems and methods may provide an unbiased interrogation of TCR specificities of an immune response, which relies on a physical interaction between the TCR and pHLA. This ligand identification method may be especially important in cancers that have low mutational burden, in which neoantigen targets may not be as prevalent compared to wildtype antigens.

As shown in FIG. 4, in certain aspects, binding predictions determined using module 1 may be validated or analyzed further, for example, using another yeast peptide display assay. This assay may use a display library where the peptides include sequences and/or sequence motifs, for example by using anchor peptides, identified in the binding prediction from the first iteration of module 1. Similarly, sequences and/or sequence motifs of interest may be identified in, for example, a public database. Such sequences may be validated using the combined wet lab and processing steps of module 1.

### IV. Module 2—Peptide Activation Predictions

The second module determines the immunogenicity of peptides to activate a particular TCR of interest, e.g., an orphan TCR. This recognizes that, though module 1 predicts peptides that bind to a TCR of interest, peptide binding does not necessarily lead to TCR activation. Thus, the second module may use T-cell/TCR activation data to train a machine learning model. Once trained, the model may be used to predict the activation potential for peptides or antigens identified by module 1.

In certain aspects, module 2 comprises wet lab steps, computational steps and/or a combination of wet lab and computational steps.

In certain aspects, module 2 uses one or more machine learning model to provide predictions of peptides that activate a TCR of interest. The machine learning model of module 2 may be trained as a global predictor of TCR-antigen activation, which is unconstrained to any specific TCR or peptide system. The global predictor may be trained using TCR-antigen activation data from assays, such as a luciferase T cell activation assay, and/or from databases of TCR-antigen activation data.

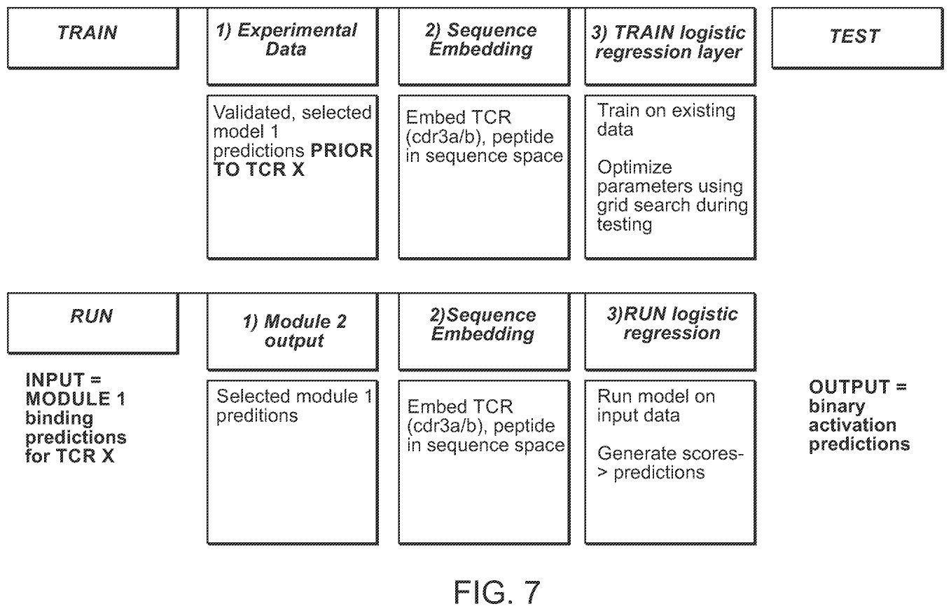

FIG. 7 provides a schematic for an exemplary module 2 of the invention. As shown, a global TCR-antigen activator prediction machine learning module is trained using data from validated module 1 peptide predictions. This training data may not be for the TCR of interest being investigated using module 2.

This validated data may include, for example, T cell activation assay data from peptides previously identified to bind to a TCR using module 1. In certain aspects, the global TCR-antigen activator prediction machine learning module is trained using receptor activation data from publicly available databases, e.g., the Immune Epitope Database and Analysis Resource (IEDB), which hosted and maintained by the U.S. National Institute of Allergy and Infectious Diseases as part of the National Institutes of Health. In certain aspects, peptide activation predictions are validated using one or more in vitro assays to assess the ability of a peptide to activate a receptor/cell. Exemplary assays to assess peptide activation include, for example, in vitro T cell coculture assays, such as antigen processing assays, target expression assays, and other methods known in the art and otherwise described herein. Data from these wet lab assays may be used to train or further train the global TCR-antigen activator prediction machine learning module.

Returning to FIG. 7, the peptide sequence of the TCR, e.g., from the cdr3a/b, is embedded, for example, in Uniprot. A logistic regression layer of the machine learning model is trained by fitting the training data a hyperparameter search with, for example, a 5× grid search. The model may be tested using a 5-fold cross validation and/or a leave-one-TCR-out validation.

As further shown in FIG. 7, after training, peptide sequences from module 1 for the TCR of interest are provided to the machine learning model. The sequence of the TCR of interest is embedded (e.g., in Uniprot). The machine learning module uses logistic regression to generate predicted probabilities of activation potential. The output from the model is a binary (yes/no) activation prediction for the peptides identified in module 1. This activation data may be used to further train the machine learning model to generate predictions for additional peptides. Alternatively or additionally, the machine learning module uses a linear regression model to predict the amount or level of activation caused by a particular peptide, peptide sequence, and/or peptide sequence motif.

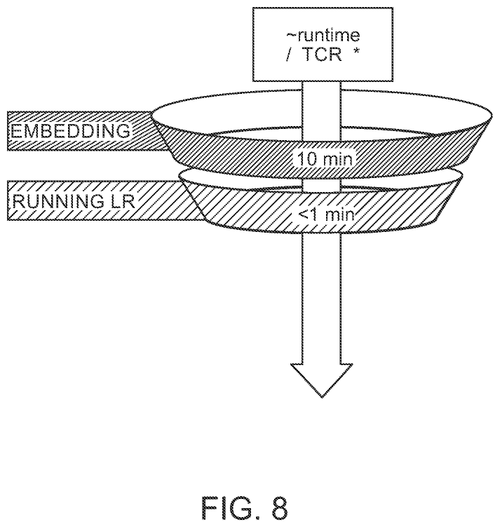

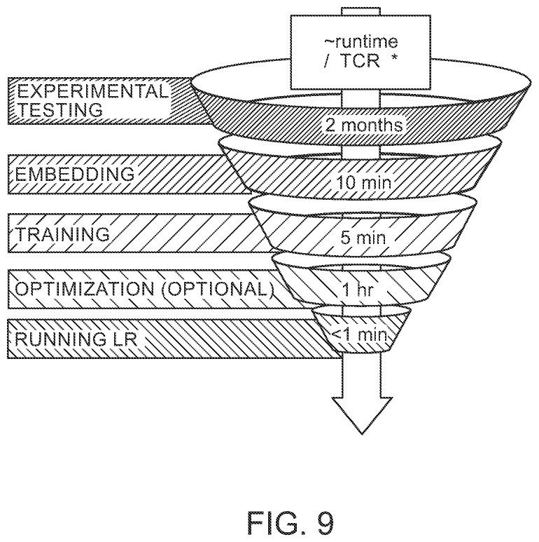

FIGS. 8 and 9 show exemplary steps used in module 2.

### V. Module 3—Exhaustive TCR Cross Reactivity Profile.

The third module is used to exhaustively profile a TCR of interest in terms of on-target and off-target cross reactivity. This module may include constructing an activation neighborhood in a latent space, which may be continually updated to reveal new activators until the cross-reactivity profile of the TCR is determined based on an absence of any new activators.

In certain aspects, module 3 incorporates one or more machine learning models to produce an exhaustive cross-reactivity profile for a TCR of interest.

In certain aspects, module 3 includes constructing a latent space representation in which peptide predictions obtained using module 1 for the TCR are embedded in a latent space. This may include both identified mimotopes from the yeast-display library and peptides (e.g., endogenous peptides) from a proteome database are correlated with the yeast-display peptides.

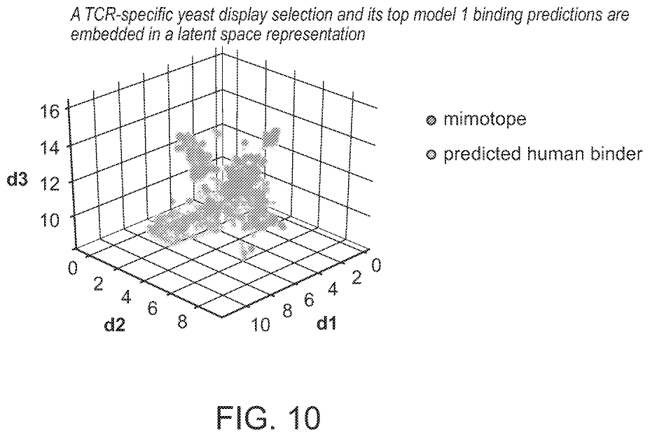

FIG. 10 shows an example of a latent space used in module 3 of the invention, in which sequences of mimotopes and predicted peptides from a proteome database (predicted human binder), which were identified using module 1, are embedded in a latent space.

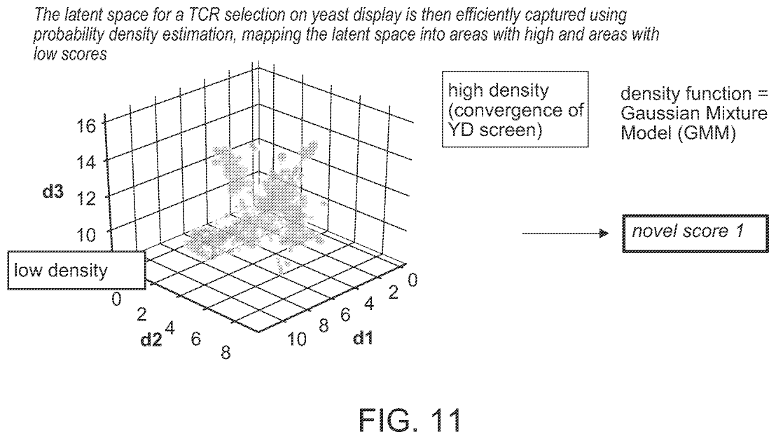

As shown in FIG. 11, the latent space for a yeast-display library selection assay is efficiently captured using probability density estimation, which maps the latent space into areas with high and low scores. Areas of high density represent converging sequences from the yeast-display library. In certain aspects, the density function incorporates a Gaussian Mixture Model (GMM).

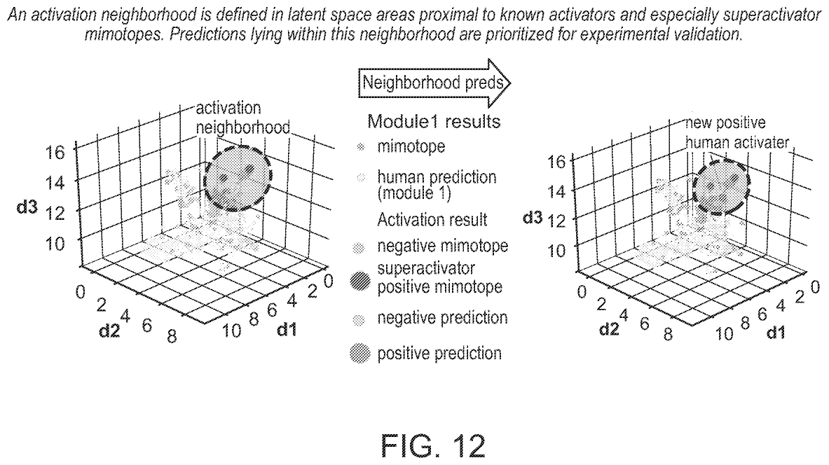

As shown in FIG. 12, an activation neighborhood is created in the latent space. This may include providing TCR/T cell activation results from module 2 for the TCR of interest and/or wet lab assays. Results for positive mimotopes (bind to TCR of interest), negative mimotopes (do not bind TCR of interest), positive and negative peptide predictions from module 2, and/or superactivator positive mimotopes are embedded in the latent space. An activation neighborhood is defined in latent space areas proximal to known activators, especially superactivator mimotopes. Predictions lying within the neighborhood are prioritized for experimental validation, e.g., using TCR/T cell activation assays. Data from these assays may be embedded in the latent space to further characterize the activation neighborhood.

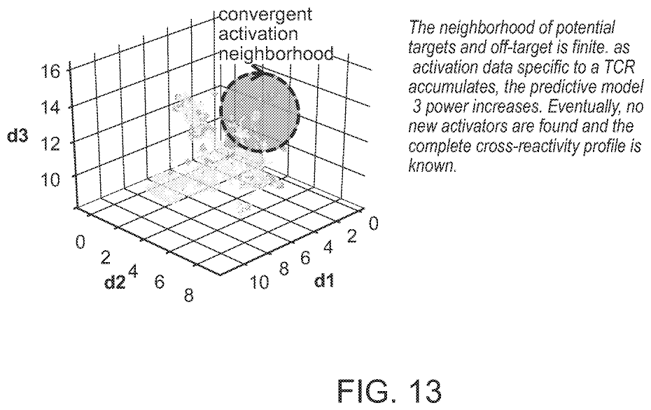

As shown in FIG. 13, iteratively repeating this process using accumulated activation data crystalizes the boundaries of the activation neighborhood. As the neighborhood becomes defined, eventually no potential new activators can be found. At this point, the cross-reactivity profile of the TCR of interest is known.

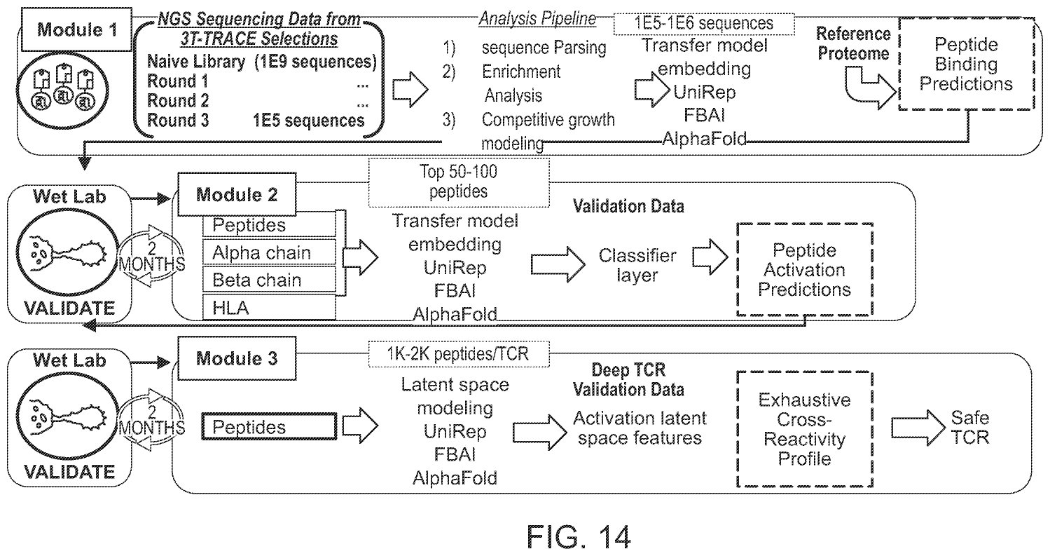

FIG. 14 provides a schematic showing an exemplary system of the invention incorporating all three modules.

As shown, model/module 1 includes a yeast-display library. The naïve library has around 10⁹ unique peptides displayed by the yeast cells. As described herein, cells/peptides from the library are selected across three rounds. By the third round, only 10⁵ unique sequences remain. Sequencing data from the peptides is obtained at each selection round. The sequences are parsed and enriched. Competitive growth modeling is for the peptide sequences across all rounds of selection. The selected sequences are compared to a reference proteome using a transfer model embedding machine learning module. The output of module 1 is peptide binding predictions.

The peptide binding predictions are validated using wet lab TCR/T cell activation assays and/or data from a database and used to train a machine learning model in model/module 2. A machine learning model in module 2 uses this data to provide peptide activation predictions. These predictions may be validated using wet lab TCR/T cell activation assays.

Peptide activation predictions from model/module 2 are provided to module 3. Module three iteratively embeds data from modules 1 and 2 onto a latent space to exhaustively profile the cross-reactivity profile for a TCR of interest.

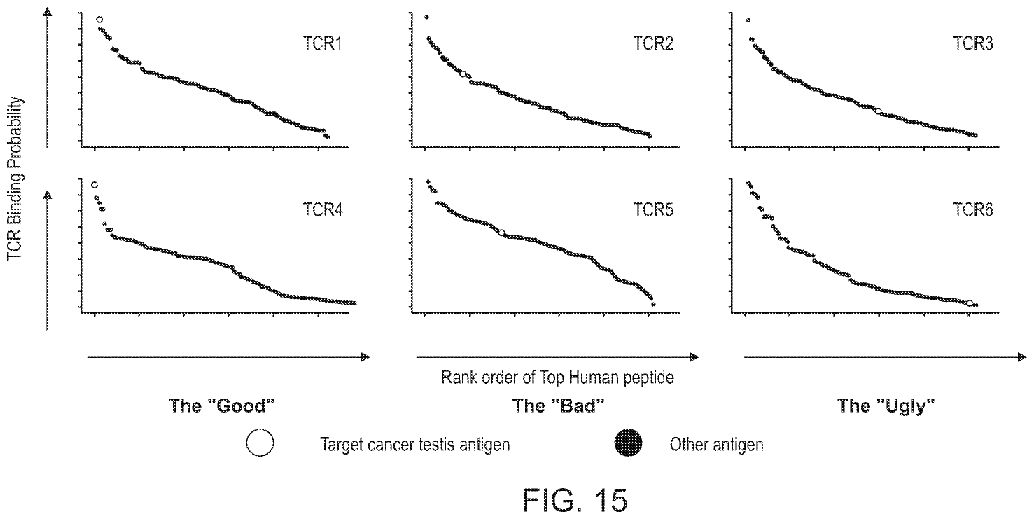

As shown in FIG. 15, the methods and systems of the invention can exhaustively profile the specificity of a TCR for antigens of interest, e.g., cancer neoantigens, and also reveal the cross-reactivity profile of the TCR for other antigens. Thus, the methods and systems of the invention effectively identify TCR that are likely candidates as the basis for specific and effective therapeutic treatments.

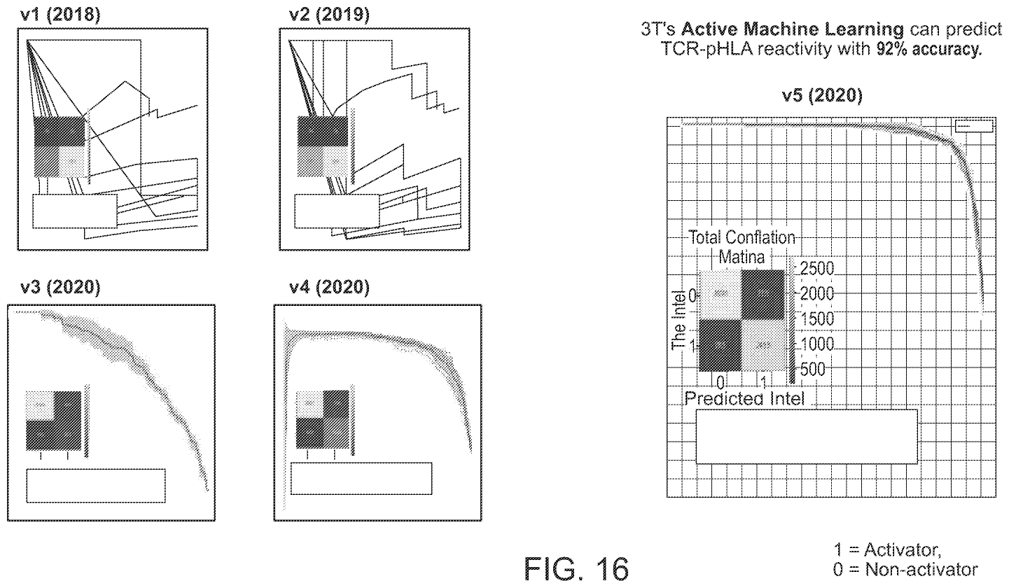

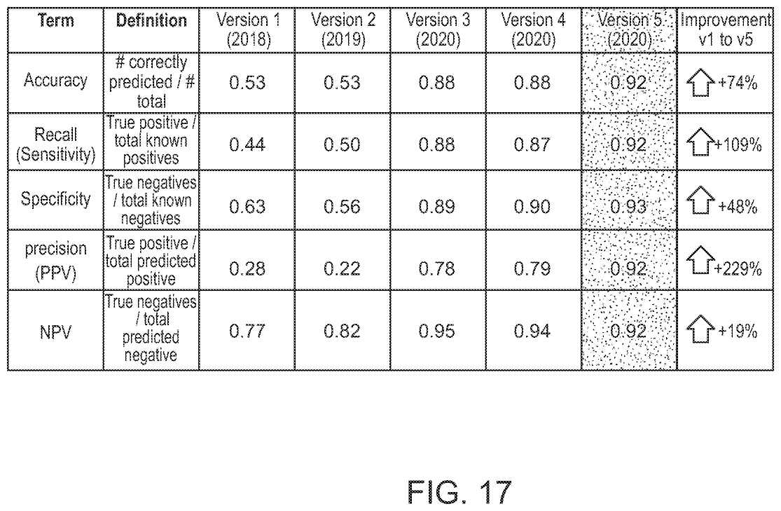

As shown in FIGS. 16 and 17, as the machine learning models in modules 1, 2, and/or 3 mature as they are trained on iterative data sets, the ability of the systems and methods of the invention to predict TCR reactivity for potential ligands steadily improves.

### VI. Exemplary Methods, Materials, and Compositions.

As explained herein, the methods and systems of the invention may incorporate pHLA yeast-display libraries, where the yeast cells display a ligand that potentially binds with a TCR of interest. In certain aspects, the peptide ligand is from about 8 to about 20 amino acids in length, usually from about 8 to about 18 amino acids, from about 8 to about 16 amino acids, from about 8 to about 14 amino acids, from about 8 to about 12 amino acids, from about 10 to about 14 amino acids, from about 10 to about 12 amino acids. It will be appreciated that a fully random library would represent an extraordinary number of possible combinations. In preferred methods, the diversity is limited at the residues that anchor the peptide to the MHC binding domains, which are referred to herein as MHC anchor residues. The position of the anchor residues in the peptide may be determined by the specific MHC binding domains. Class I binding domains can have anchor residues at the P2 position, and at the last contact residue. Class II binding domains have an anchor residue at P1, and depending on the allele, at one of P4, P6 or P9. For example, the anchor residues for IE^{k} are P1 {l,L,V} and P9 {K}; the anchor residues for HLA-DR15 are P1 {l,L,V} and P4 {F, Y}. Anchor residues for DR alleles are shared at P1, with allele-specific anchor residues at P4, P6, P7, and/or P9.

In some embodiments, the binding domains of a major histocompatibility complex protein may be soluble domains of Class II alpha and beta chain. In certain aspects, the binding domains are subjected to mutagenesis and selected for amino acid changes to enhance the solubility of the single chain polypeptide, without changing the peptide binding contacts. In certain specific embodiments, the binding domains are HLA-DR4a comprising the set of amino acid changes {M36L, V132M}; and HLA-DR4|3 comprising the set of amino acid changes {H62N, D72E}. In certain specific embodiments, the binding domains are HLA-DR15a comprising the set of amino acid changes {F12S, M23K}; and HLA-DR15|3 comprising the amino acid change {P11 S}. In certain specific embodiments, the binding domains are H2 IE^{k}a comprising the set of amino acid changes {18T, F12S, L14T, A56V} and H2 IE^{k}|3 comprising the set of amino acid changes {W6S, L8T, L34S}.

In some embodiments, the binding domains of a major histocompatibility complex protein comprise the alpha 1 and alpha2 domains of a Class I MHC protein, which are provided in a single chain with β2 microglobulin. In some such embodiments the Class I protein has been subjected to mutagenesis and selected for amino acid changes that enhance the solubility of the single chain polypeptide, without altering the peptide binding contacts. In certain specific embodiments, the binding domains are HLA-A2 alpha 1 and alpha 2 domains, comprising the amino acid change {Y84A}. In certain specific embodiments, the binding domains are H2-L^{d} alpha 1 and alpha 2 domains, comprising the amino acid change {M31 R}. In certain specific embodiments the binding domains are HLA-B57 alpha 1, alpha 2 and alpha 3 domains, comprising the amino acid change {Y84A}.

Major histocompatibility complex proteins (also called human leukocyte antigens, HLA, or the H2 locus in the mouse) are protein molecules expressed on the surface of cells that confer a unique antigenic identity to these cells. MHC/HLA antigens are target molecules that are recognized by T-cells and natural killer (NK) cells as being derived from the same source of hematopoietic reconstituting stem cells as the immune effector cells (“self) or as being derived from another source of hematopoietic reconstituting cells (“non-self). Two main classes of HLA antigens are recognized: HLA class I and HLA class II.

The MHC proteins used in the libraries and methods of the invention may be from any mammalian or avian species, e.g., primate sp., particularly humans; rodents, including mice, rats and hamsters; rabbits; equines, bovines, canines, felines; etc. Of particular interest are the human HLA proteins, and the murine H-2 proteins. Included in the HLA proteins are the class II subunits HLA-DPa, HLA-DPβ, HLA-DQα, HLA-DQβ, HLA-DRα and HLA-DRβ, and the class I proteins HLA-A, HLA-B, HLA-C, and β₂-microglobulin. Included in the murine H-2 subunits are the class I H-2K, H-2D, H-2L, and the class II I-Aα, I-Aβ, 1-Eα and I-Eβ, and β₂-microglobulin.

The MHC binding domains are typically a soluble form of the normally membrane-bound protein. The soluble form is derived from the native form by deletion of the transmembrane domain. Conveniently, the protein may be truncated, removing both the cytoplasmic and transmembrane domains. In some embodiments, the binding domains of a major histocompatibility complex protein are soluble domains of Class II alpha and beta chain. In some such embodiments the binding domains have been subjected to mutagenesis and selected for amino acid changes that enhance the solubility of the single chain polypeptide, without altering the peptide binding contacts.

An “allele” is one of the different nucleic acid sequences of a gene at a particular locus on a chromosome. One or more genetic differences can constitute an allele. An important aspect of the HLA gene system is its polymorphism. Each gene, MHC class I (A, B and C) and MHC class II (DP, DQ, and DR) exists in different alleles. Current nomenclature for HLA alleles is designated by numbers, as described by Marsh et al.: Nomenclature for factors of the HLA system, 2010. Tissue Antigens 75:291-455, herein specifically incorporated by reference. For HLA protein and nucleic acid sequences, see Robinson et al. (2011), The IMGT/HLA database. Nucleic Acids Research 39 Suppl 1:D1 171-6, herein specifically incorporated by reference.

The numbering of amino acid residues on the various MHC proteins and variants may be made to be consistent with the full-length polypeptide. Boundaries may be offset to either be the end of the MHC peptide binding domain (as judged by examining crystal structures) for the ‘mini’ MHCs, and the end of the Beta2/Alpha2/Alpha3 domains as judged by structure and/or sequence for the ‘full length’ MHCs.

The function of MHC molecules is to bind peptide fragments derived from pathogens and display them on the cell surface for recognition by the appropriate T cells. Thus, T cell receptor recognition can be influenced by the MHC protein that is presenting the antigen. The term MHC context refers to the recognition by a TCR of a given peptide, when it is presented by a specific MHC protein.

Class II binding domains generally comprise the a1 and a2 domains for the α chain, and the β1 and β2 domains for the β chain. Not more than about 10, usually not more than about 5, preferably none of the amino acids of the transmembrane domain will be included. The deletion will be such that it does not interfere with the ability of the α2 or β2 domain to bind peptide ligands.

In some embodiments, the binding domains of a major histocompatibility complex protein are soluble domains of Class II alpha and beta chain. In some such embodiments the binding domains have been subjected to mutagenesis and selected for amino acid changes that enhance the solubility of the single chain polypeptide, without altering the peptide binding contacts.

In certain specific embodiments, the binding domains are an HLA-DR allele. The HLA-DRA binding domains can be combined with any one of the HLA-DRB binding domains. In certain such embodiments, the HLA-DRA allele is paired with the binding domains of an HLA-DRB4 allele. The HLA-DRB4 allele can be selected from the publicly available DRB4 alleles. In other such embodiments the HLA-DRA allele is paired with the binding domains of an HLA-DRB15 allele. The HLA-DRB15 allele can be selected from the publicly available DRB15 alleles. In other embodiments the Class II binding domains are an H2 protein, e.g., I-Aα, I-Aβ, 1-Ea and I-Eβ. In some such embodiments, the binding domains are H2 IE^{k}a which may comprise the set of amino acid changes {I8T, F12S, L14T, A56V}; and H2 IE^{k}|3 which may comprise the set of amino acid changes {W6S, L8T, L34S}.

Class I HLA/MHC. For class I proteins, the binding domains may include the a1, a2 and a3 domain of a Class I allele, including without limitation HLA-A, HLA-B, HLA-C, H-2K, H-2D, H-2L, which are combined with β₂-microglobulin. Not more than about 10, usually not more than about 5, preferably none of the amino acids of the transmembrane domain will be included. The deletion will be such that it does not interfere with the ability of the domains to bind peptide ligands.

In certain specific embodiments, the binding domains are HLA-A2 binding domains, e.g., comprising at least the alpha 1 and alpha 2 domains of an A2 protein. A large number of alleles have been identified in HLA-A2, including without limitation HLA-A*02:01:01:01 to HLA-A*02:478, which sequences are available at, for example, Robinson et al. (2011), The IMGT/HLA database. Nucleic Acids Research 39 Suppl 1:D1 171-6. Among the HLA-A2 allelic variants, HLA-A*02:01 is the most prevalent. The binding domains may comprise the amino acid change {Y84A}.

In certain specific embodiments, the binding domains are HLA-B57 binding domains, e.g., comprising at least the alpha 1 and alpha 2 domains of a B57 protein. The HLA-B57 allele can be selected from the publicly available B57 alleles.

T cell receptor refers to the antigen/MHC binding heterodimeric protein product of a vertebrate, e.g., mammalian, TCR gene complex, including the human TCR α, β, γ and δ chains. For example, the complete sequence of the human β TCR locus has been sequenced, as published by Rowen et al. (1996) Science 272(5269): 1755-1762; the human a TCR locus has been sequenced and resequenced, for example see Mackelprang et al. (2006) Hum Genet. 119(3):255-66; see a general analysis of the T-cell receptor variable gene segment families in Arden Immunogenetics, 1995; 42(6):455-500; each of which is herein specifically incorporated by reference for the sequence information provided and referenced in the publication.

The multimerized T cell receptor for selection in module 1 may be a soluble protein comprising the binding domains of a TCR of interest, e.g., TCRα/β, TCRy/δ. The soluble protein may be a single chain, or more usually a heterodimer. In some embodiments, the soluble TCR is modified by the addition of a biotin acceptor peptide sequence at the C terminus of one polypeptide. After biotinylation at the acceptor peptide, the TCR can be multimerized by binding to biotin binding partner, e.g., avidin, streptavidin, traptavidin, neutravidin, etc. The biotin binding partner can comprise a detectable label, e.g., a fluorophore, mass label, etc., or can be bound to a particle, e.g., a paramagnetic particle. Selection of ligands bound to the TCR can be performed by flow cytometry, magnetic selection, and the like as known in the art.

Peptide ligands of the TCR are peptide antigens against which an immune response involving T lymphocyte antigen specific response can be generated. Such antigens include antigens associated with autoimmune disease, infection, foodstuffs such as gluten, etc., allergy or tissue transplant rejection. Antigens also include various microbial antigens, e.g., as found in infection, in vaccination, etc., including but not limited to antigens derived from virus, bacteria, fungi, protozoans, parasites and tumor cells. Tumor antigens include tumor specific antigens, e.g. immunoglobulin idiotypes and T cell antigen receptors; oncogenes, such as p21/ras, p53, p210/bcr-abl fusion product; etc.; developmental antigens, e.g. MART-1/Melan A; MAGE-1, MAGE-3; GAGE family; telomerase; etc.; viral antigens, e.g. human papilloma virus, Epstein Barr virus, etc.; tissue specific self-antigens, e.g. tyrosinase; gp100; prostatic acid phosphatase, prostate specific antigen, prostate specific membrane antigen; thyroglobulin, a-fetoprotein; etc.; and self-antigens, e.g. her-2/neu; carcinoembryonic antigen, muc-1, and the like.

Conventional methods of assembling the coding sequences can be used in the methods and systems of the invention. In order to generate the diversity of peptide ligands, randomization, error prone PCR, mutagenic primers, and the like as known in the art are used to create a set of polynucleotides. The library of polynucleotides is typically ligated to a vector suitable for the host cell of interest. In various embodiments the library is provided as a purified polynucleotide composition encoding the P- L₁-β-L₂-a-L₃-T polypeptides; as a purified polynucleotide composition encoding the P-L β-L₂-a-L₃-T polypeptides operably linked to an expression vector, where the vector can be, without limitation, suitable for expression in yeast cells; as a population of cells comprising the library of polynucleotides encoding the P-L β-L₂-a-L₃-T polypeptides, where the population of cells can be, without limitation yeast cells, and where the yeast cells may be induced to express the polypeptide library.

The term “specificity” refers to the proportion of negative test results that are true negative test result. Negative test results include false positives and true negative test results.

The term “sensitivity” is meant to refer to the ability of an analytical method to detect small amounts of analyte. Thus, as used here, a more sensitive method for the detection of amplified DNA, for example, would be better able to detect small amounts of such DNA than would a less sensitive method. “Sensitivity” refers to the proportion of expected results that have a positive test result.

The term “reproducibility” as used herein refers to the general ability of an analytical procedure to give the same result when carried out repeatedly on aliquots of the same sample.

Sequencing platforms that can be used in the present disclosure include but are not limited to: pyrosequencing, sequencing-by-synthesis, single-molecule sequencing, second-generation sequencing, nanopore sequencing, sequencing by ligation, or sequencing by hybridization. Preferred sequencing platforms are those commercially available from Illumina (RNA-Seq) and Helicos (Digital Gene Expression or “DGE”). “Next generation” sequencing methods include, but are not limited to those commercialized by: 1) 454/Roche Lifesciences including but not limited to the methods and apparatus described in Margulies et al., Nature (2005)437:376-380 (2005); and U.S. Pat. Nos. 7,244,559; 7,335,762; 7,211,390; 7,244,567; 7,264,929; 7,323,305; 2) Helicos Biosciences Corporation (Cambridge, MA) as described in U.S. application Ser. No. 11/167,046, and U.S. Pat. Nos. 7,501,245; 7,491,498; 7,276,720; and in U.S. Patent Application Publication Nos. US20090061439; US20080087826; US20060286566; US20060024711; US20060024678; US20080213770; and US20080103058; 3) Applied Biosystems (e.g. SOLiD sequencing); 4) Dover Systems (e.g., Polonator G. 007 sequencing); 5) Illumina as described U.S. Pat. Nos. 5,750,341; 6,306,597; and 5,969,119; and 6) Pacific Biosciences as described in U.S. Pat. Nos. 7,462,452; 7,476,504; 7,405,281; 7,170,050; 7,462,468; 7,476,503; 7,315,019; 7,302,146; 7,313,308; and US Application Publication Nos. US20090029385; US20090068655; US20090024331; and US20080206764. All references are herein incorporated by reference. Such methods and apparatuses are provided here by way of example and are not intended to be limiting.

Expression construct: Sequences encoding a peptide disclosed herein or a TCR disclosed herein may be introduced on an expression vector, e.g., into a cell to be engineered, as a vaccine, etc. The TCR sequence may be introduced at the site of the endogenous gene, e.g., using CRISPR technology (see, for example Eyquem et al. (2017) Nature 543:113-117; Ren et al. (2017) Protein & Cell 1-10).

Amino acid sequence variants are prepared by introducing appropriate nucleotide changes into the coding sequence, as described herein. Such variants represent insertions, substitutions, and/or specified deletions of, residues as noted. Any combination of insertion, substitution, and/or specified deletion is made to arrive at the final construct, provided that the final construct possesses the desired biological activity as defined herein.

The nucleic acid encoding the sequence is inserted into a vector for expression and/or integration. Many such vectors are available. For example, the CRISPR/Cas9 system can be directly applied to human cells by transfection with a plasmid that encodes Cas9 and sgRNA. The viral delivery of CRISPR components has been extensively demonstrated using lentiviral and retroviral vectors. Gene editing with CRISPR encoded by non-integrating virus, such as adenovirus and adenovirus-associated virus (AAV), has also been reported. Recent discoveries of smaller Cas proteins have enabled and enhanced the combination of this technology with vectors that have gained increasing success for their safety profile and efficiency, such as AAV vectors.

The vector components generally include, but are not limited to, one or more of the following: an origin of replication, one or more marker genes, an enhancer element, a promoter, and a transcription termination sequence. Vectors include viral vectors, plasmid vectors, integrating vectors, and the like.

The sequences may be produced recombinantly as a fusion polypeptide with a heterologous polypeptide, e.g., a signal sequence or other polypeptide having a specific cleavage site at the N-terminus of the mature protein or polypeptide. In general, the signal sequence may be a component of the vector, or it may be a part of the coding sequence that is inserted into the vector. The heterologous signal sequence selected preferably is one that is recognized and processed (i.e., cleaved by a signal peptidase) by the host cell. In mammalian cell expression the native signal sequence may be used, or other mammalian signal sequences may be suitable, such as signal sequences from secreted polypeptides of the same or related species, as well as viral secretory leaders, for example, the herpes simplex gD signal.

Expression vectors may contain a selection gene, also termed a selectable marker.

This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Expression vectors will contain a promoter that is recognized by the host organism and is operably linked to the coding sequence. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Transcription from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus (such as murine stem cell virus), hepatitis-B virus and most preferably Simian Virus 40 (SV40), from heterologous mammalian promoters, e.g., the actin promoter, PGK (phosphoglycerate kinase), or an immunoglobulin promoter, or from heat-shock promoters, provided such promoters are compatible with the host cell systems. The early and late promoters of the SV40 virus are conveniently obtained as an SV40 restriction fragment that also contains the SV40 viral origin of replication.

Transcription by higher eukaryotes is often increased by inserting an enhancer sequence into the vector. Enhancers are cis-acting elements of DNA, usually about from 10 to 300 bp in length, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, a-fetoprotein, and insulin). Typically, however, one will use an enhancer from a eukaryotic virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence, but is preferably located at a site 5′ from the promoter.

Expression vectors for use in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

Suitable host cells for cloning or expressing the DNA in the vectors herein are the prokaryotic, yeast, or other eukaryotic cells described above. Examples of useful mammalian host cell lines are mouse L cells (L-M[TK-], ATCC #CRL-2648), monkey kidney CV1 line transformed by SV40 (COS-7, ATCC CRL 1651); human embryonic kidney line (293 or 293 cells subcloned for growth in suspension culture); baby hamster kidney cells (BHK, ATCC CCL 10); Chinese hamster ovary cells/-DHFR (CHO); mouse Sertoli cells (TM4); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2).

Host cells, including engineered T cells, etc. can be transfected with the above-described expression vectors. Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (suchasinsulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression, and will be apparent to the ordinarily skilled artisan.

In another embodiment of the invention, an article of manufacture containing materials useful for the treatment of the conditions described above is provided. The article of manufacture comprises a container and a label. Suitable containers include, for example, bottles, vials, syringes, and test tubes. The containers may be formed from a variety of materials such as glass or plastic. The container holds a composition that is effective for treating the condition and may have a sterile access port (for example the container may be an intravenous solution bag or a vial having a stopper pierceable by a hypodermic injection needle). The active agent in the composition can be a vector suitable for introducing the sequence into a targeted cell for expression. The label on or associated with the container indicates that the composition is used for treating the condition of choice. Further container(s) may be provided with the article of manufacture which may hold, for example, a pharmaceutically-acceptable buffer, such as phosphate-buffered saline, Ringer's solution or dextrose solution. The article of manufacture may further include other materials desirable from a commercial and user standpoint, including other buffers, diluents, filters, needles, syringes, and package inserts with instructions for use.

Also provided herein in certain aspects are libraries of polypeptides comprising or consisting essentially of at least one of the SCT polypeptides of the present disclosure and/or at least one of the polypeptide compositions of the present disclosure. In certain aspects, the libraries are peptide-HLA-B*35 libraries. It will be appreciated that a fully random library would represent an extraordinary number of possible combinations. In preferred methods, the target peptides (i.e., peptide ligands) of the library are diversified (e.g., randomized or not randomized) at multiple positions, and the diversity is limited at the residues that anchor the peptide to the MHC binding domains, which are referred to herein as MHC anchor residues. The position of the anchor residues in the peptide are determined by the specific MHC binding domains. HLA-B*35 binding domains have anchor residues at the P2 position, and at the last contact residue (e.g., the P9 position). In certain aspects, the target peptide (i.e., peptide ligand) of the SCT polypeptides have NNK codons at positions 1, 3-8 were used to diversity the peptide, and known anchor residues position 2 and position 9 were restricted to allowed amino acids. In certain aspects, the libraries comprise SCT polypeptides comprising HIV(Pol448-456), β2 microgrobulin, and an HLA-B*35 alpha chain. In certain aspects, the libraries comprise SCT polypeptides comprising NY-ESO-1 [e.g., NY-ESO-1(94-102)], β2 microgrobulin, and an HLA-B*35 alpha chain.

In certain aspects, the library comprises at least 10⁶, at least 10⁷, more usually at least 10⁸, or at least 10⁹ different target peptides (i.e., peptide ligands) that are displayed on cell surface in the context of the HLA-B*35 allele. In some certain, the libraries can be used to identify the recognition properties of ligands of HLA-B*35-restricted T cell receptors.

The different target peptides (i.e., peptide ligands) of the libraries may be created by any methods known in the art, including error prone mutagenesis, and a gene editing system, e.g., clustered, regularly interspaced, short, palindromic repeats (CRISPR)/CRISPR-associated (Cas) system, transcription activator-like effector nucleases (TALEN) system, zinc-finger protein (ZNF) system, or Transposase system into cells.

Further provided herein in certain embodiments are pharmaceutical compositions comprising or consisting essentially of at least one of the SCT polypeptides of the present disclosure and/or at least one of the polypeptide compositions of the present disclosure.

Also provided herein in certain embodiments are cells comprising or consisting essentially of at least one of the SCT polypeptides of the present disclosure and/or at least one of the polypeptide compositions of the present disclosure. In some embodiments, the cells are yeast cells, e.g., Saccharomyces cerevisiae cells. In other embodiments, the cells are mammalian cells or insect cells.

In some embodiments, a target peptide is displayed on a cell surface by modifying the cell with the SCT polypeptides or the SCT polypeptide compositions of the present disclosure. Such modification of the cell with the SCT polypeptides or the SCT polypeptide compositions may be performed by a number of methods well known in the art, including, but not limited to, transfection, electroporation, recombination, transformation, transduction, or CRISPR gene editing.

In some embodiments, expression of the SCT polypeptides or the SCT polypeptide compositions is induced in the cells. Inducing expression of the SCT polypeptides or the SCT polypeptide compositions may be achieved by methods well known in the art, including inducing cell proliferation, expressing the SCT polypeptides or the SCT polypeptide compositions under an inducible promoter, targeting promotor sequences, or gene editing.

Further provided herein in certain embodiments are first nucleic acids comprising or consisting essentially of a second nucleic acid encoding at least one of the SCT polypeptides of the present disclosure and/or at least one of the polypeptide compositions of the present disclosure.

Also provided herein in certain embodiments are expression vectors comprising or consisting essentially of at least one of the nucleic acids of the present disclosure. In some embodiments, the nucleic acids of the present disclosure are located under an inducible promoter in the expression vector, such that the expression of the nucleic acids is inducible.

Further provided herein in certain embodiments are kits comprising or consisting essentially of a first container comprising the pharmaceutical compositions of the present disclosure in solution or in lyophilized form, optionally, a second container containing a diluent or reconstituting solution for the lyophilized formulation and instructions for (i) use of the solution or (ii) reconstitution and/or use of the lyophilized composition form.

Also provided herein in certain embodiments are methods comprising or consisting essentially of preparing one or more polypeptides selected from the group consisting of the SCT polypeptides of the present disclosure and the polypeptide compositions of the present disclosure, the method comprising co-expressing protein disulfide isomerase with one or more of the polypeptides of the present disclosure, culturing the cells of the present disclosure, and isolating the one or more polypeptides from the cell or a culture medium thereof.

In some embodiments, disulfide bond formation can be enhanced with co-expression of protein disulfide isomerase (PDI).

Further provided herein in certain embodiments are methods of displaying a target peptide on a cell surface, the method comprising modifying the cell with a first nucleic acid comprising or consisting essentially of a second nucleic acid encoding at least one of the SCT polypeptides and/or at least one of the polypeptide compositions of the present disclosure. Modifying the cell with the SCT polypeptides or the polypeptide compositions may be performed by a number of methods well known in the art, including, but not limited to, transfection, electroporation, recombination (e.g., homologous recombination), transformation, transduction, or gene editing (e.g., introducing a CRISPR-Cas9 system, a TALEN system, or a ZNF system into cells). An exemplary gene editing system comprises a nuclease and a guide RNA. A CRISPR system comprises a CRISPR nuclease (e.g., CRISPR (clustered regularly interspaced short palindromic repeats)-associated (Cas) endonuclease or a variant thereof, such as Cas9) and a guide RNA. A CRISPR nuclease associates with a guide RNA that directs nucleic acid cleavage by the associated endonuclease by hybridizing to a recognition site in a polynucleotide. The guide RNA comprises a direct repeat and a guide sequence, which is complementary to the target recognition site. In certain embodiments, the CRISPR system further comprises a tracrRNA (trans-activating CRISPR RNA) that is complementary (fully or partially) to the direct repeat sequence present on the guide RNA. As used herein, a “TALEN” nuclease is an endonuclease comprising a DNA-binding domain comprising a plurality of TAL domain repeats fused to a nuclease domain or an active portion thereof from an endonuclease or exonuclease, including but not limited to a restriction endonuclease, homing endonuclease, and yeast HO endonuclease. A “zinc finger nuclease” or “ZFN” refers to a chimeric protein comprising a zinc finger DNA-binding domain fused to a nuclease domain from an endonuclease or exonuclease, including but not limited to a restriction endonuclease, homing endonuclease, and yeast HO endonuclease.

In some embodiments, the methods optionally include inducing expression of the SCT polypeptides and/or the at least one of the polypeptide compositions by, for example, inducing cell proliferation, expressing the SCT polypeptides or the SCT polypeptide compositions under an inducible promoter and activating the promotor, targeting promotor sequences, or gene editing. In some embodiments, the cells are yeast cells, e.g., Saccharomyces cerevisiae cells. In some embodiments, the cells are mammalian cells or insect cells.

Further provided herein in certain embodiments are kits comprising or consisting essentially of a first container comprising the pharmaceutical compositions of the present disclosure in solution or in lyophilized form, optionally, a second container containing a diluent or reconstituting solution for the lyophilized formulation and instructions for (i) use of the solution or (ii) reconstitution and/or use of the lyophilized composition form.

Also provided herein in certain embodiments are in vitro methods for producing activated T cells, comprising or consisting essentially of contacting T cells with one or more of the SCT polypeptides of the present disclosure and/or one or more of the polypeptide compositions of the present disclosure.

Further provided herein in certain embodiments are activated T cells, produced by the methods of the present disclosure, that selectively recognize a cell expressing one or more peptides selected from the group consisting of the target peptides of the present disclosure.

Sequencing platforms that can be used in the present disclosure include but are not limited to: pyrosequencing, sequencing-by-synthesis, single-molecule sequencing, second-generation sequencing, nanopore sequencing, sequencing by ligation, or sequencing by hybridization. Preferred sequencing platforms are those commercially available from Illumina (RNA-Seq) and Helicos (Digital Gene Expression or “DGE”). “Next generation” sequencing methods include, but are not limited to those commercialized, for example, by: 1) 454/Roche Lifesciences including but not limited to the methods and apparatus described in Margulies et al., Nature (2005) 437:376-380 (2005); and U.S. Pat. Nos. 7,244,559; 7,335,762; 7,211,390; 7,244,567; 7,264,929; 7,323,305; 2) Helicos BioSciences Corporation (Cambridge, Mass.) as described in U.S. application Ser. No. 11/167,046, and U.S. Pat. Nos. 7,501,245; 7,491,498; 7,276,720; and in U.S. Patent Publication Nos. US20090061439; US20080087826; US20060286566; US20060024711; US20060024678; US20080213770; and US20080103058; 3) Applied Biosystems (e.g. SOLiD sequencing); 4) Dover Systems (e.g., Polonator G.007 sequencing); 5) Illumina as described U.S. Pat. Nos. 5,750,341; 6,306,597; and 5,969,119; and 6) Pacific Biosciences as described in U.S. Pat. Nos. 7,462,452; 7,476,504; 7,405,281; 7,170,050; 7,462,468; 7,476,503; 7,315,019; 7,302,146; 7,313,308; and US Patent Publication Nos. US20090029385; US20090068655; US20090024331; and US20080206764.

The results of any of modules 1, 2, or 3 can be used to engineer antigen-presenting cells. Antigen-presenting cells (APCs) may include cells that present complexes formed between HLA antigens and the peptides on its surface. APCs may be obtained by contacting the peptides, or the nucleotides encoding the peptides, and can be prepared from subjects who are the targets of treatment and/or prevention, and can be administered as vaccines by themselves or in combination with other drugs including the peptides, exosomes, or cytotoxic T cells. The APCs are not limited to any kind of cells and includes dendritic cells (DCs), Langerhans cells, macrophages, B cells, and activated T cells, all of which are known to present proteinaceous antigens on their cell surface so as to be recognized by lymphocytes. Since DC is a representative APC having the strongest CTL inducing action among APCs, DCs find particular use as the APCs.

Cells may be engineered to express a TCR of interest, such as a TIL, or to respond to a peptide antigen provided herein. A number of different cell types are suitable for engineering, particularly T cells or NK cells. In some embodiments the cells for engineering are autologous. In some embodiments the cells are allogeneic.

A T cell stimulated against any of the peptides disclosed herein can be used as vaccines similar to the peptides. Thus, the present invention provides isolated T cells that are stimulated by any of the present peptides. Such T cells can be obtained by (1) administering to a subject or (2) contacting (stimulating) subject-derived APCs, and CD8-positive cells, or peripheral blood mononuclear leukocytes in vitro with the peptide. T cells, which have been stimulated by stimulation from APCs that present the peptides, can be derived from subjects who are targets of treatment and/or prevention, and can be administered by themselves or in combination with other drugs including the peptides or exosomes for the purpose of regulating effects. The obtained T cells act specifically against target cells presenting the peptides, for example, the same peptides used for priming. The target cells can be cells that express endogenously, or cells that are transfected with genes, and cells that present the peptides on the cell surface due to stimulation by these peptides can also become targets of attack.

In certain aspects, the engineered cell is a T cell. The term “T cells” refers to mammalian immune effector cells that may be characterized by expression of CD3 and/or T cell antigen receptor, which cells can be engineered to express a TCR provided herein or stimulated to respond to a peptide provided herein. In some embodiments the T cells are selected from naive CD8⁺ T cells, cytotoxic CD8⁺ T cells, naive CD4⁺ T cells, helper T cells, e.g., T_{H}1, T_{H}2, T_{H}9, T_{H}11, T_{H}22, T_{FH}; regulatory T cells, e.g., T_{R}1, natural T_{Reg}, inducible T_{Reg}; memory T cells, e.g., central memory T cells, T stem cell memory cells (T_{S}CM)-effector memory T cells, NKT cells, γδ T cells. In some embodiments, the engineered cells comprise a complex mixture of immune cells, e.g., tumor infiltrating lymphocytes (TILs) isolated from an individual in need of treatment. In certain aspects, T cells are contacted with a peptide in vitro, i.e., where the T cells are then transferred to a recipient.

Effector cells may include autologous or allogeneic immune cells having cytolytic activity against a target cell, including without limitation tumor cells. Effector cells may be obtained by engineering peripheral blood lymphocytes (PBL) in vitro, then culturing with a cytokine and/or antigen combination that increases activation. The cells may be optionally separated from non-desired cells prior to culture, prior to administration, or both. Cell-mediated cytolysis of target cells by immunological effector cells is believed to be mediated by the local directed exocytosis of cytoplasmic granules that penetrate the cell membrane of the bound target cell.

Cytotoxic T lymphocytes (CTL) reactive to tumor cells are specific effector cells for adoptive immunotherapy and are of interest for engineering by priming with peptides disclosed herein, or engineering to express a TCR disclosed herein. Induction and expansion of CTL is antigen-specific and MHC restricted.

T cells collected from a subject may be separated from a mixture of cells by techniques that enrich for desired cells, or may be engineered and cultured without separation. An appropriate solution may be used for dispersion or suspension. Such solution will generally be a balanced salt solution, e.g., normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

Techniques for affinity separation may include magnetic separation, using antibody-coated magnetic beads, affinity chromatography, cytotoxic agents joined to a monoclonal antibody or used in conjunction with a monoclonal antibody, e.g., complement and cytotoxins, and “panning” with antibody attached to a solid matrix, e.g., a plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g., propidium iodide). Any technique may be employed which is not unduly detrimental to the viability of the selected cells. The affinity reagents may be specific receptors or ligands for the cell surface molecules indicated above. In addition to antibody reagents, peptide-MHC antigen and T cell receptor pairs may be used; peptide ligands and receptor; effector and receptor molecules, and the like.

The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum (FCS).

The collected and optionally enriched cell population may be used immediately for genetic modification, or may be frozen at liquid nitrogen temperatures and stored, being thawed and capable of being reused. The cells will usually be stored in 10% DMSO, 50% FCS, 40% RPMI 1640 medium.

The engineered cells may be infused to the subject in any physiologically acceptable medium by any convenient route of administration, normally intravascularly, although they may also be introduced by other routes, where the cells may find an appropriate site for growth. Usually, at least 1×10⁶ cells/kg will be administered, at least 1×10⁷ cells/kg, at least 1×10⁸ cells/kg, at least 1×10⁹ cells/kg, at least 1×10¹⁰ cells/kg, or more, usually being limited by the number of T cells that are obtained during collection.

The peptide and T cell receptor sequences are also useful in screening assays for patient samples, where a T cell containing sample from an individual, e.g., a blood sample, tumor biopsy sample, lymph node sample, bone marrow sample, etc. is analyzed for (i) the presence of T cells comprising a TCR identified herein, and/or (ii) the presence of T cells response to a peptide described herein. The determination of the presence of T cells may be made according to any convenient method, e.g., determining stimulation by measuring proliferation, etc., in response to the presence of the peptide in an HLA complex, or as presented by an APC. The presence of a specific TCR may be determined by sequencing of mRNA, sequencing of genomic DNA, etc. The presence of T cells responsive to the peptide or having a TCR of interest allows the patient to be assigned to a group that can be treated by vaccination, APC transfer, etc. with that group.

The systems and methods of the invention, including one or more of modules 1, 2, and 3 may incorporate one or more computer-based systems, which may refer to a hardware means, a software means, and/or a data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention. Such presentation provides a skilled artisan with a ranking of similarities and identifies the degree of similarity contained in the test expression repertoire.

The computer-based systems, including machine learning models used in the invention, may be implemented in hardware or software, or a combination of both. In one embodiment of the invention, a machine-readable storage medium is provided, the medium comprising a data storage material encoded with machine readable data which, when using a machine programmed with instructions for using said data, is capable of displaying any of the datasets and data comparisons of this invention. In some embodiments, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program can be implemented in a high-level procedural or object-oriented programming language to communicate with a computer system. However, the programs can be implemented in assembly or machine language, if desired. In any case, the language may be a compiled or interpreted language. Each such computer program can be stored on a storage media or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

Further provided herein is a method of storing and/or transmitting, via computer, sequence, module predictions, and/or other data collected by the methods disclosed herein. Any computer or computer accessory including, but not limited to software and storage devices, can be utilized to practice the present invention. Sequence or other data can be input into a computer by a user either directly or indirectly. Additionally, any of the devices which can be used to sequence DNA or analyze DNA or analyze peptide binding data can be linked to a computer, such that the data is transferred to a computer and/or computer-compatible storage device. Data can be stored on a computer or suitable storage device (e.g., CD). Data can also be sent from a computer to another computer or data collection point via methods well known in the art (e.g., the internet, ground mail, air mail). Thus, data collected by the methods described herein can be collected at any point or geographical location and sent to any other geographical location.

As described herein, modules 1, 2, and/or 3 may include the use of one or more machine learning (ML) models in the systems and methods of the invention. This includes not only systems and methods using ML, but also the training of ML systems to increase their accuracy and predictive value.

Machine learning is branch of computer science in which machine-based approaches are used to make predictions. (Bera et al., Nat Rev Clin Oncol., 16(11):703-715 (2019)). ML-based approaches involve a system learning from data fed into it, and using this data to make and/or refine predictions. Id. Machine learning is distinct from traditional, rule-based or statistics-based program models. (Rajkomar et al., N Engl J Med, 380:1347-58 (2019)). Rule-based program models require software engineers to code explicit rules, relationships, and correlations. Id. For example, in the medical context, a physician may input a patient's symptoms and current medications into a rule-based program. In response, the program will provide a suggested treatment based upon preconfigured rules.

In contrast, and as a generalization, in ML a model learns from examples fed into it. Id Over time, the ML model learns from these examples and creates new models and routines based on acquired information. Id. As a result, an ML model may create new correlations, relationships, routines or processes never contemplated by a human. A subset of ML is deep learning (DL). (Bera et al. (2019)). DL uses artificial neural networks. ADL network generally comprises layers of artificial neural networks. Id. These layers may include an input layer, an output layer, and multiple hidden layers.Id. DL has been shown to learn and form relationships that exceed the capabilities of humans. (Rajkomar et al. (2019)).

By combining the ability of ML, including DL, to develop novel routines, correlations, relationships and processes amongst TCR-peptide binding and/or activation data features, the methods and systems of the disclosure can provide an exhaustive cross-reactivity profile for a TCR of interest, including orphan TCRs.

Any suitable machine learning system may be used in modules 1, 2, and 3. For example, the machine learning systems may learn in a supervised manner, an unsupervised manner, a semi-supervised manner, or through reinforcement learning.

In supervised learning models, the machine learning system is given training data categorized as input variables paired with output variables from which to learn patterns and make inferences in order to generate a prediction on previously unseen test data. Supervised models replicate an identified mapping system and recognize and respond to patterns in data without explicit instructions. Supervised models are advantageous for performing classification tasks, in which data inputs are separated into categories. Supervised models are also advantageous for regression tasks, in which the output variable is a real value, such as a price or a volume. The accuracy of a supervised model is easy to evaluate, because there is a known output variable to which the model is optimizing.

In an unsupervised model or autonomous model, the machine learning system is only given input training data without paired output data from which to identify patterns autonomously. Unsupervised models identify underlying patterns or structures in training data to make predictions for test data. Unsupervised models are advantageous for clustering data, anomaly detection, and for independently discovering rules for data. The accuracy of unsupervised models is harder to evaluate because there is no predefined output variable to which the system is optimizing. Autonomous models may employ periods of both supervised and unsupervised learning in order to optimize predictions.

In semi-supervised models, the machine learning system is given training data comprising input variables, with output variable pairs available for only a limited pool of the input variables. The model uses the input variables with known output variables and the remaining input training data to learn patterns and make inferences in order to generate a prediction on previously unseen test data. A semi-supervised model may query the user for additional paired output data based on unlabeled data.

In a reinforcement learning model, the machine learning system is given neither input variables nor output variables. Rather, the model provides a “reward” condition and then seeks to maximize the cumulative reward condition by trial and error. A common reinforcement learning model is a Markov Decision Process.

A common supervised learning model is a “decision tree.” Decision trees are non-parametric supervised learning models that use simple decision rules to infer a classification for test data from the features in the test data. In classification trees, test data take a finite set of values, or classes, whereas in regression trees, the test data can take continuous values, such as real numbers. Decision trees have some advantages in that they are simple to understand and can be visualized as a tree starting at the root (usually a single node) and repeatedly branch to the leaves (multiple nodes) that are associated with the classification. See Criminisi, 2012, Decision Forests: A unified framework for classification, regression, density estimation, manifold learning and semi-supervised learning, Foundations and Trends in Computer Graphics and Vision 7(2-3):81-227, incorporated by reference.

Another supervised learning model is a “support-vector machine” (SVM) or “support-vector network.” SVMs are supervised learning models for classification and regression problems. When used for classification of new data into one of two categories, such binding/not binding a TCR or activating/not activating a TCR, an SVM creates a hyperplane in multidimensional space that separates data points into one category or the other. Although the original problem may be expressed in terms that require only finite dimensional space, linear separation of data between categories may not be possible in finite dimensional space. Consequently, multidimensional space is selected to allow construction of hyperplanes that afford clean separation of data points. See Press, W. H. et al., Section 16.5. Support Vector Machines, Numerical Recipes: The Art of Scientific Computing (3rd ed.). New York: Cambridge University (2007), incorporated herein by reference. Where output variables are unavailable for input variables in the training data, SVMs can be designed as unsupervised learning models using support vector clustering. See Ben-Hur, 2001, Support Vector Clustering, J Mach Learning Res 2:125-137, incorporated by reference.

Some models rely on clustering training data and test data to find patterns and make predictions. A “k-nearest neighbor” (k-NN) model is a non-parametric supervised learning model for classification and regression problems. A k-nearest neighbor model assumes that similar data exists in close proximity, and assigns a category or value to each data point based on the k nearest data points. k-NN models may be advantageous when the data has few outliers and can be defined by homogeneous features. A common unsupervised learning model that uses clustering is a “k-means” clustering model. A k-means model looks to find clusters of data in input data and test data. K-means models are advantageous when a defined number of clusters are known to exist in the data and are also advantageous when the test data has few outliers and can be defined homogeneous features. Additional models that cluster training data include, for example, farthest-neighbor, centroid, sum-of-squares, fuzzy k-means, and Jarvis-Patrick clustering.

Bayesian algorithms can also be used to find patterns in training and test data to make predictions. Bayesian networks are probabilistic graphical models that represent a set of random variables and their conditional dependencies via directed acyclic graphs (DAGs). The DAGs have nodes that represent random variables that may be observable quantities, latent variables, node unknown parameters or hypotheses.Edges represent conditional dependencies; nodes that are not connected represent variables that are conditionally independent of each other. Each is associated with a probability function that takes, as input, a particular set of values for the node's parent variables, and gives (as output) the probability (or probability distribution, if applicable) of the variable represented by the node.

Regression analysis is another statistical process that can be used to find patterns in training and test data to make predictions. It includes techniques for modeling and analyzing relationships between multiple variables. Specifically, regression analysis focuses on changes in a dependent variable in response to changes in single independent variables. Regression analysis can be used to estimate the conditional expectation of the dependent variable given the independent variables. The variation of the dependent variable may be characterized around a regression function and described by a probability distribution. Parameters of the regression model may be estimated using, for example, least squares methods, Bayesian methods, percentage regression, least absolute deviations, nonparametric regression, or distance metric learning.

Trained machine learning models can become “stable learners.” A stable learner is a model that is less sensitive to perturbation of predictions based on new training data. Stable learners can be advantageous where test data is stable, but can be less advantageous where the system needs to continually improve performance to accurately predict new test data.

Several machine learning system types can be combined into final predictive models known as ensembles. Ensembles can be divided into two types, homogenous ensembles and heterogeneous ensembles. Homogenous ensembles combine multiple machine learning models of the same type. Heterogeneous ensembles combine multiple machine learning models of different types. Ensembles can provide the advantage of being more accurate than any of the individual member models (“members”) in the ensemble. The number of members combined in an ensemble may impact the accuracy of a final prediction. Accordingly, it is advantageous to determine the optimal number of members when designing an ensemble system.

Ensembles may combine or aggregate outputs from individual members using “voting”-type methods for classification systems and “averaging”-type methods for regression systems.

In a “majority voting” method, each member makes a prediction for test data and the prediction that receives more than half of the votes is the final output for the ensemble. If none of the predictions receives more than half of the votes, it may be determined that the ensemble is unable to make a stable prediction. In a “plurality voting” method the most voted prediction, even if receiving less than half of the votes, may be considered the final output for the ensemble. In a “weighted voting” method, the votes of more accurate members are multiplied by a weight afforded each member based on its accuracy.

In a “simple averaging” method, each member makes a prediction for test data and the average of the outputs is calculated. This method reduces overfit and can be advantageous in creating smoother regression models. In a “weight averaging” method, the prediction output of each member is multiplied by a weight afforded each member based on its accuracy. Voting methods, averaging methods, and weighted methods can be combined to improve the accumcy of ensembles.

Members within an ensemble can each be trained independently or new members can be trained utilizing information from previously trained members. In a “parallel ensemble”, the ensemble seeks to provide greater accuracy than individual members by exploiting the independence between members, for example, by training multiple members simultaneously and aggregating the outputs from members. In “sequential ensemble systems”, the ensemble seeks to provide greater accuracy than individual members by exploiting the dependence between members, for example, by utilizing information from a first member to improve the training of a second member and weighting outputs from members.

Overall accuracy for ensembles can also be optimized by using ensemble meta-algorithms, for example a “bagging” algorithm to reduce variance, a “boosting” algorithm to reduce bias, or a “stacking” algorithm to improve predictions.

Boosting algorithms reduce bias and can be used to improve less accurate, or “weak learning” models. A member may be considered a “weak learning” model if it has a substantial error rate, but its performance is non-random, for example an error rate of 0.5 for binary classifications. Boosting algorithms incrementally build the ensemble by training each member sequentially with the same training data set, examining prediction errors for test data, and assigning weights to training data based on the difficulty for members to make an accurate prediction. In each sequential member trained, the algorithm emphasizes training data that previous members found difficult. Members are then weighted based on the accuracy of their prediction outputs in view of the weight applied to their training data. The predictions from each member may be combined by weighted voting-type or weighted averaging-type methods. Boosting algorithms are advantageous when combining multiple weak learning models. Boosting algorithms may, however, result in over-fitting test data to training data.

Examples of boosting algorithms include AdaBoost, gradient boosting, eXtreme Gradient Boost (XGBoost). See Freund, 1997, A decision-theoretic generalization of on-line learning and an application to boosting, J Comp Sys Sci 55:119; and Chen, 2016, XGBoost: A Scalable Tree Boosting System, arXiv:1603.02754, both incorporated by reference.

Bagging algorithms or “bootstrap aggregation” algorithms reduce variance by averaging together multiple estimates from members. Bagging algorithms provide each member with a random sub-sample of a full training data set, with each random sub-sample known as a “bootstrap” sample. In the bootstrap samples, some data from the training data set may appear more than once and some data from the training data set may not be present Because sub-samples can be generated independently from one another, training can be done in parallel. The predictions for test data from each member are then aggregated, such as by voting-type or averaging-type methods.

An example of a bagging algorithm that may be utilized is a “random forest”. In a random forest the ensemble combines multiple randomized decision tree models. Each decision tree model is trained from a bootstrap sample from a training set. The training set itself may be a random subset of features from an even larger training set. By providing a random subset of the larger training set at each split in the learning process, spurious correlations that can results from the presence of individual features that are strong predictors for the response variable are reduced. By averaging predictions for test data, variance of the ensemble decreases resulting in an improved prediction. Random forests may be autonomous models and may include periods of both supervised and unsupervised learning. Bagging may be less advantageous in optimizing an ensemble combining stable learning systems, since stable learning systems tend provide generalized outputs with less variability over the bootstrap samples. See Breiman, 2001, Random Forests, Machine Learning 45:5-32, incorporated by reference.

Stacking algorithms or “stacked generalization” algorithms improve predictions by using a meta-machine learning model to combine and build the ensemble. In stacking algorithms, base member models are trained with a training dataset and generate as an output a new dataset. This new dataset is then used as a training dataset for the meta-machine learning model to build the ensemble. Stacking algorithms are generally advantageous when building heterogeneous ensembles.

Neural networks, modeled on the human brain, allow for processing of information and machine learning. Neural networks include nodes that mimic the function of individual neurons, and the nodes are organized into layers. Neural networks include an input layer, an output layer, and one or more hidden layers that define connections from the input layer to the output layer. Systems and methods of the invention may include any neural network that facilitates machine learning. The system may include a known neural network architecture, such as GoogLeNet (Szegedy, et al. Going deeper with convolutions, in CVPR 2015, 2015); AlexNet (Krizhevsky, et al. Imagenet classification with deep convolutional neural networks, in Pereira, et al. Eds., Advances in Neural Information Processing Systems 25, pages 1097-3105, Curran Associates, Inc., 2012); VGG16 (Simonyan & Zisserman, Very deep convolutional networks for large-scale image recognition, CoRR, abs/3409.1556, 2014); or FaceNet (Wang et al., Face Search at Scale: 80 Million Gallery, 2015), each of the aforementioned references are incorporated by reference.

Deep learning neural networks (also known as deep structured learning, hierarchical learning or deep machine learning) include a class of machine learning operations that use a cascade of many layers of nonlinear processing units for feature extraction and transformation. Each successive layer uses the output from the previous layer as input. The algorithms may be supervised or unsupervised and applications include pattern analysis (unsupervised) and classification (supervised). Certain embodiments are based on unsupervised learning of multiple levels of features or representations of the data. Higher level features are derived from lower-level features to form a hierarchical representation. Those features are preferably represented within nodes as feature vectors. Deep learning by the neural network includes learning multiple levels of representations that correspond to different levels of abstraction; the levels form a hierarchy of concepts. In some embodiments, the neural network includes at least 5 and preferably more than ten hidden layers. The many layers between the input and the output allow the system to operate via multiple processing layers.

Within the network, nodes are connected in layers, and signals travel from the input layer to the output layer. Each node in the input layer may correspond to a respective one of the features from the training data. The nodes of the hidden layer are calculated as a function of a bias term and a weighted sum of the nodes of the input layer, where a respective weight is assigned to each connection between a node of the input layer and a node in the hidden layer. The bias term and the weights between the input layer and the hidden layer are learned autonomously in the training of the neural network. The network may include thousands or millions of nodes and connections. Typically, the signals and state of artificial neurons are real numbers, typically between 0 and 1. Optionally, there may be a threshold function or limiting function on each connection and on the unit itself, such that the signal must surpass the limit before propagating. Back propagation is the use of forward stimulation to modify connection weights, and is sometimes done to train the network using known correct outputs. See WO 2016/182551, U.S. Pub. 2016/0174902, U.S. Pat. No. 8,639,043, and U.S. Pub. 2017/0053398, each incorporated by reference.

Deep learning is part of a broader family of machine learning methods based on learning representations of data. An observation can be represented in many ways such as a vector of intensity values per pixel, or in a more abstract way as a set of edges, regions of particular shape, etc. Those features are represented at nodes in the network. Preferably, each feature is structured as a feature vector, a multi-dimensional vector of numerical features that represent some object. The feature provides a numerical representation of objects, since such representations facilitate processing and statistical analysis. Feature vectors are similar to the vectors of explanatory variables used in statistical procedures such as linear regression. Feature vectors are often combined with weights using a dot product in order to construct a linear predictor function that is used to determine a score for making a prediction.

The vector space associated with those vectors may be referred to as the feature space. In order to reduce the dimensionality of the feature space, dimensionality reduction may be employed. Higher-level features can be obtained from already available features and added to the feature vector, in a process referred to as feature construction. Feature construction is the application of a set of constructive operators to a set of existing features resulting in construction of new features.

For example, a convolutional neural network (CNN) is a class of deep neural network generally designed for two-dimensional image inputs in which a signal travels from the input layer through hidden layers comprising “convolutional layers” and “fully connected layers” to the output layer. In the input layer, each pixel from a signal is mapped to a node. The input layer is connected to a convolutional layer. In a convolutional layer, each node is “sparsely connected”, that is connected to only a sub-matrix of nodes from the previous layer. The connection between the submatrix of nodes and the convolutional layer is subject to a bias term as a set of weights designed detect a given feature in the input. The submatrix and weights together are known as a “filter,” “kernel,” or “feature detector”. For a given convolutional layer, each filter is the same size and shape and applies the same set of weights. Each node in the convolutional layer is provided a summary of the weighted information from the filter as a scalar dot product. The filters are staggered from one another and may overlap such that each node in convolution layer provides a weighted summary for a different sub-matrix from the previous layer. A threshold function may be applied to each node in the convolution layer to determine whether the node will propagate the information from the filter, a function known as “squashing.”

Sliding the filter systematically across the entire input allows the filter to discover a given feature anywhere in the input. The function of sliding the filter over entire image can be controlled by the number of nodes over which the filter movies, known as the “stride” of the convolutional layer. The stride determines the distance that each filter is staggered from adjacent filters and the degree of overlap between filters. The final two-dimensional array of dot products of the convolutional layer is known as the “convolved feature,” “activation map,” or “feature map.”

In some instances, it may also be convenient to “pad” an input to a convolutional layer with zero values around the border of the input, a process known as zero-padding. Zero-padding allows the size of feature maps to be controlled. This can allow for the feature map to remain the same size as the input through multiple layers of the CNN. The function of adding zero-padding is known as “wide-convolution” versus “narrow convolution” when no zero-padding is added.

The use of multiple convolutional layers in the network allows for hierarchical decomposition of the input. Convolutional filters that operate directly on input values may learn to extract low level features, such as lines. Convolutional filters that operate on the output from earlier convolution layers may learn to extract features that are combinations of lower-level features.

A CNN may also comprise nonlinear layers (ReLU) and/or pooling or sub sampling layers. A ReLU layer receives a feature map and replaces any negative values in the feature map with a zero. The purpose of the ReLU layer is to introduce non-linearity into the CNN and is advantageous when the input data that the CNN is expected to learn and identify is non-linear. The non-linear output map from a ReLU is known as a “rectified” feature map. A pooling layer reduces the size of the feature map or rectified feature map through dimensionality reduction in a process known as “spatial pooling,” “subsampling,” or downsampling.” For example, each node in a pooling layer may be connected to a sub-matrix of nodes from a convolution or ReLU layer. Each node in the pooling layer may then provide, for example, only the highest value, average of, or sum of the values in each submatrix. Pooling layers can be advantageous to make input representations smaller and more manageable, reduce the number of parameters and computations in the network, reduce the impact of distortions in the input image, and help scale representation of the image. This may reduce training time and control overfitting in the CNN.

The final output from the convolutional, ReLU, and/or pooling layers, is provided to a fully connected layer. The fully connected layers operate under the same principles as a traditional neural network. In a fully connected layer, each node in the layer is connected to all of the nodes in a previous layer and all of the nodes in a succeeding layer. The purpose of a fully connected layer is to classify the features extracted by the convolutional layers, for example using single vector machines (SVM).

Backpropagation in CCNs involves adjusting the weights of filters based on the error rate of the CNN, known as “loss.” During backpropagation, the CNN determines the estimated loss at every node in each convolutional layer and adjusts filter weights accordingly to minimize loss. A CNN may be trained by multiple rounds of backpropagation.

A deconvolutional neural network (DNN) is another class of deep neural network designed to generate an image from a feature map or from the output from a CNN. A DNN learns and makes predictions as to the pooling, ReLU, and convolution layers that a feature map may have undergone and performs the opposite function, e.g., unpooling and deconvolution.

The systems and methods of the disclosure may use fully convolutional networks (FCN). In contrast to CNNs, FCNs can learn representations locally within a data set, and therefore, can detect features that may occur sparsely within a data set.

The systems and methods of the disclosure may use recurrent neural networks (RNN). RNNs have an advantage over CNNs and FCNs in that they can store and learn from inputs over multiple time periods and process the inputs sequentially.

The systems and methods of the disclosure may use generative adversarial networks (GAN), which find particular application in training neural networks. One network is fed training exemplars from which it produces synthetic data. The second network evaluates the agreement between the synthetic data and the original data. This allows GANs to improve the prediction model of the second network.

The features detected by the machine learning system may be any quantity, structure, pattern, or other element that can be measured from the training data. Features may be unrecognizable to the human eye. Features may be created autonomously by the machine learning system. Alternatively, features may be created with user input.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

We claim:

1. A method of profiling a TCR or TCRm, the method comprising:
(a) conducting one or more analyses to obtain a plurality of peptide binding predictions that are representative of peptide-HLA targets that a TCR binds;
(b) conducting one or more analyses using the peptide binding predictions to obtain a plurality of peptide activation predictions that are representative of target peptides identified from step (a) that are predicted to activate the TCR; and
(c) conducting one or more analyses using the peptide binding predictions and the peptide activation predictions to obtain a cross-reactivity profile of the TCR with respect to the target peptides identified from step (b) to thereby obtain a profile of the TCR.

2. The method of claim 1, wherein the step of conducting one or more analyses to obtain a plurality of peptide binding predictions comprises:
contacting a screening library of cells with a TCR or other macromolecule having one or more antigen binding domains, wherein different cells of the screening library express a different randomized peptide antigen; and
identifying a peptide sequence for a plurality of the randomized peptide antigens of the screening library that bind to the TCR or other macromolecule having one or more antigen binding domains.

3. The method of claim 2, wherein the method further comprises expanding cells of the screening library having randomized peptide antigen that binds with the TCR or other macromolecule.

4. The method of claim 2, wherein the method further comprises embedding the sequences of the randomized peptide antigens that bind to the TCR or other macromolecule onto a latent space defined by molecular interactions of peptide antigens and antigen binding domains.

5. The method of claim 4, further comprising identifying clusters of mapped randomized peptide antigen sequences and performing a probability position matrix on the sequences of each cluster.

6. The method of claim 4, further comprising embedding the peptide activation predictions onto the latent space and identifying areas of the latent space proximal to peptide activation predictions for known TCR activators.

7. The method of claim 6, further comprising identifying a plurality of embedded peptide sequences in an area of the latent space proximal to the peptide activation prediction for one or more known TCR activators.

8. The method of claim 6, wherein the step of conducting one or more analyses using the peptide binding predictions and the peptide activation predictions to obtain a cross-reactivity profile comprises identifying embedded peptide sequences of the randomized peptide antigens in an area of the latent space proximal to the peptide activation predictions for known TCR activators.

9. A method for treating a subject having a cancer that comprises cancer cells that display a target peptide, the method comprising:
providing to the subject a therapeutic TCR that binds the target peptide to thereby treat the subject, wherein the therapeutic TCR was identified by a process comprising:
(a) conducting one or more analyses to obtain a plurality of peptide binding predictions that are representative of the target peptide to which the TCR binds;
(b) conducting one or more analyses using the peptide binding predictions to obtain a plurality of peptide activation predictions that are representative of target peptides identified from step (a) that are predicted to activate the TCR;
(c) conducting one or more analyses using the peptide binding predictions and the peptide activation predictions to obtain a cross-reactivity profile of the TCR with respect to the target peptides identified from step (b) to thereby obtain a profile of the TCR; and
(d) identifying the therapeutic TCR using the cross-reactivity profile of the TCR with respect.

10. The method of claim 9, wherein the step of conducting one or more analyses to obtain a plurality of peptide binding predictions comprises:
contacting a screening library of cells with a TCR or other macromolecule having one or more antigen binding domains, wherein different cells of the screening library express a different randomized peptide antigen; and
identifying a peptide sequence for a plurality of the randomized peptide antigens of the screening library that bind to the TCR or other macromolecule having one or more antigen binding domains.

11. The method of claim 10, wherein the method further comprises expanding cells of the screening library having randomized peptide antigen that binds with the TCR or other macromolecule.

12. The method of claim 10, wherein the method further comprises embedding the sequences of the randomized peptide antigens that bind to the TCR or other macromolecule onto a latent space defined by molecular interactions of peptide antigens and antigen binding domains.

13. The method of claim 12, further comprising identifying clusters of mapped randomized peptide antigen sequences and performing a probability position matrix on the sequences of each cluster.

14. The method of claim 13, further comprising identifying mapped randomized peptide antigen sequences that have a peptide sequence similar or identical to a peptide sequence of the target peptide.

15. The method of claim 14, further comprising embedding the peptide activation predictions onto the latent space and identifying areas of the latent space proximal to peptide activation predictions for known TCR activators.

16. The method of claim 15, further comprising identifying a plurality of embedded peptide sequences in an area of the latent space proximal to the peptide activation prediction for one or more known TCR activators.

17. The method of claim 16, wherein the step of conducting one or more analyses using the peptide binding predictions and the peptide activation predictions to obtain a cross-reactivity profile comprises identifying embedded peptide sequences of the randomized peptide antigens in an area of the latent space proximal to the peptide activation predictions for known TCR activators.

18. A method for preparing a composition comprising a TCR useful for treating cancer in a subject, the method comprising:
(a) obtaining tumor tissue from a subject and extracting tumor infiltrating lymphocytes from the tumor tissue;
(b) conducting one or more analyses to obtain a plurality of peptide binding predictions that are representative of peptide-HLA targets in the tumor tissue that bind to a TCR of an extracted tumor infiltrating lymphocyte;
(c) conducting one or more analyses using the peptide binding predictions to obtain a plurality of peptide activation predictions that are representative of target peptides identified from step (a) that are predicted to activate the TCR;
(d) conducting one or more analyses using the peptide binding predictions and the peptide activation predictions to obtain a cross-reactivity profile of the TCR with respect to the target peptides identified from step (b) to thereby obtain a profile of the TCR; and
(e) causing engineered immune cells to express the TCR.

19. The method of claim 18, wherein the step of conducting one or more analyses to obtain a plurality of peptide binding predictions comprises:
contacting a screening library of cells with the TCR, wherein different cells of the screening library express a different peptide antigen, which has a peptide sequence of a neoantigen, a wildtype peptide, a spliced peptide, a human endogenous retrovirus (hERV), an aberrantly expressed tumor specific antigen (aeTSA), from a frameshift mutation, from a gene fusion, from an alternative splicing mutation, from aberrant translation, and/or from an alternative promoter sequence expressed by a cell; and
identifying a peptide sequence for a plurality of the different peptide antigens of the screening library that bind to the TCR.

20. The method of claim 19, wherein the method further comprises expanding cells of the screening library having a peptide antigen that binds with the TCR.

21. The method of claim 19, wherein the method further comprises embedding the sequences of the randomized peptide antigens that bind to the TCR onto a latent space defined by molecular interactions of the peptide antigens and an antigen binding domain of the TCR.

22. The method of claim 21, further comprising identifying clusters of mapped peptide antigen sequences and performing a probability position matrix on the sequences of each cluster.

23. The method of claim 22, further comprising embedding the peptide activation predictions onto the latent space and identifying areas of the latent space proximal to peptide activation predictions for known TCR activators.

24. The method of claim 23, further comprising identifying a plurality of embedded peptide sequences in an area of the latent space proximal to the peptide activation prediction for one or more known TCR activators.

25. The method of claim 24, wherein the step of conducting one or more analyses using the peptide binding predictions and the peptide activation predictions to obtain a cross-reactivity profile comprises identifying embedded peptide sequences of the different peptide antigens in an area of the latent space proximal to the peptide activation predictions for known TCR activators.
